(19) United States
(12) Patent Application Publication
(10) Pub. No.: US 2018/0148470 A1
(43) Pub. Date: May 31, 2018

(54) CHOLIC ACID DERIVATIVE, AND PREPARATION METHOD AND MEDICAL USE THEREOF

(71) Applicants: Jiangsu Hansoh Pharmaceutical Group Co., Ltd., Jiangsu (CN); Shanghai Hansoh Biomedical Co., Ltd., Shanghai (CN)

(72) Inventors: Chenghai LI, Shanghai (CN); Rudi BAO, Shanghai (CN); Aifeng LV, Jiangsu (CN); Huijuan ZHONG, Jiangsu (CN)

(21) Appl. No.: 15/569,918

(22) PCT Filed: Apr. 13, 2016
(86) PCT No.: PCT/CN2016/079167

§ 371 Date: Oct. 27, 2017

(30) Foreign Application Priority Data
Apr. 28, 2015 (CN) 201510204748.8

(51) Int. Cl. C07J 41/00; C07J 9/00; C07J 51/00; C07J 31/00; C07J 43/00

(52) U.S. Cl. CPC C07J 41/0061; C07J 9/005; C07J 43/003; C07J 51/00; C07J 31/006; C07J 41/0094

(57) ABSTRACT

The present invention relates to a cholic acid derivative, and a preparation method and a medical use thereof. In particular, the present invention relates to a cholic acid derivative of formula (I), a stereoisomer, or a pharmaceutically acceptable salt thereof. The series of compounds can be used to treat FXR mediated diseases, including cardiovascular disease, atherosclerosis, arteriosclerosis, hypercholesterolemia, hyperlipidemia and chronic hepatitis diseases, chronic liver diseases, gastrointestinal diseases, nephrosis, heart vascular diseases, metabolic diseases, cancer (for example colorectal cancer) or neurological diseases, such as strokes and other diseases, with a wide range of medical applications, and are expected to develop into a new generation of FXR agonists.

### FIELD OF THE INVENTION

The present invention relates to the field of pharmaceutical synthesis, and specifically relates to a cholic acid derivative, and a preparation method and use thereof.

### BACKGROUND OF THE INVENTION

The farnesyl derivative X receptor (FXR) is a member of the hormone nuclear receptor superfamily, which is mainly expressed in the liver, small intestine, kidney and adrenal gland, and is less expressed in adipose tissue and the heart. It is named FXR, because farnesol was initially thought to be its ligand. When the FXR ligand directly binds to the ligand-binding domain (LBD) of the carboxy terminus of FXR, the conformation of the nuclear receptor changes and forms a heterodimer with the retinal derivative receptor (RXR), which ultimately integrates with the specific FXR DNA response element of a target gene to regulate the transcription of the target gene, and is also involved in the regulation of sugar and lipid metabolism. FXR is thus an important energy regulator. Primary bile acid, chenodeoxycholic acid, is the most effective ligand for FXR, and secondary bile acid, lithocholic acid and deoxycholic acid, can also activate FXR. There are also synthetic FXR ligands (such as 6-ECDCA, GW4064, etc.), which are several times stronger than natural ligands in binding FXR. The main target genes of FXR include bile salt export pump (BSEP), bile acid binding protein (IBABP) and small heterodimeric partner receptor (SHP), etc. FXR regulates the expression of these genes by reacting with FXR reaction elements (FXRE) on these gene promoters. However, there is no typical FXR binding response sequence in the promoter sequence of certain major FXR regulatory genes, such as cholesterol 7α hydroxylase (CYP7α1), so FXR blocks the transcription of CYP7α1 and other LRH-1 target genes by indirectly inducing the expression of transcriptional repressor SHP, which then forms inhibitory complexes with the liver receptor homologue (LRH-1) of CYP7α1 promoter. FXR plays an important role in bile acid and cholesterol metabolism, which ha caused more and more attention to be drawn to its relationship with liver-related diseases. Finding a new FXR agonist has become a hot research topic in the treatment of liver diseases, including cholestasis syndrome. As a treatment for primary biliary cirrhosis (PBC), 6-ethyl chenodeoxycholic acid (obeticholic acid) has completed Phase III clinical trials, and will be marketed as early as 2015. This compound also shows satisfactory treatment effects in non-alcoholic steatohepatitis. FXR agonists can either increase bile acid-dependent bile flow by stimulating bile salt export pump (BSEP), or can reduce cholestasis by stimulating MRP2 to increase non-bile acid-dependent bile flow. FXR is expected to be a target for screening new drugs that can treat other metabolic diseases, including cholestatic diseases, nonalcoholic steatohepatitis, and the like. Furthermore, current studies have shown that FXR agonists may be valuable in the treatment and research of diseases including atherosclerosis, cholestatic diseases caused by bile acid disorder, liver fibrosis, cirrhosis, cancer and the like (please refer to Table 1).

TABLE 1
Diseases that may benefit from FXR agonists

arteriosclerosis
bile acid disorders

benign intrahepatic cholestasis
progressive familial intrahepatic cholestasis (type 1, 2 and 3)
primary biliary cirrhosis
primary biliary cirrhosis, primary sclerosing cholangitis

cancer
cholesterol gallstone
diabetes
dyslipidemia
fibrosis diseases

chronic hepatitis (type B and C type)
non-viral hepatitis

inflammatory bowel disease
alteration of intestinal flora
liver transplantation
non-alcoholic fatty liver disease and non-alcoholic steatohepatitis

### SUMMARY OF THE INVENTION

During the course of research, the inventors found a series of cholic acid derivatives as represented by formula (I). These compounds have a significant agonistic effect on FXR activity, and can be used in the treatment of FXR mediated diseases comprising cardiovascular disease, atherosclerosis, arteriosclerosis, hypercholesterolemia, hyperlipidemia and chronic hepatitis diseases, chronic liver diseases, gastrointestinal diseases, nephrosis, heart vascular diseases, metabolic diseases, cancer (for example colorectal cancer) or neurological indications, such as strokes and other diseases, with a wide range of medical applications, and are expected to be developed into a new generation of FXR agonists.

In one aspect, the present invention provides a cholic acid derivatives of formula (I), a stereoisomer or a pharmaceutically acceptable salt thereof:

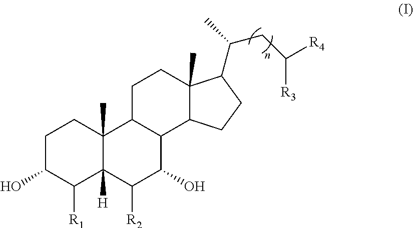

wherein,

R₁, R₂ and R₃ are each selected from the group consisting of hydrogen, fluorine, bromine, trifluoromethyl and C₁₋₈ alkyl;

R₄ is selected from the group consisting of:

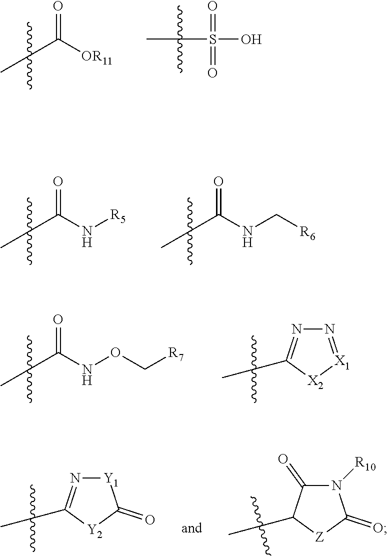

X₁ is selected from the group consisting of nitrogen and CR₈;

X₂ is selected from the group consisting of CR₈R₉, NR₁₀, oxygen and sulfur atom;

Y₁ and Y₂ are each independently selected from the group consisting of CR₈R₉, NR₁₀, oxygen and sulfur atom;

Z is selected from the group consisting of CR₈R₉, NR₁₀, oxygen and sulfur atom;

R₅ is selected from the group consisting of hydrogen, hydroxy, cyano, amino, C₁₋₈ alkoxy and SO₂R₁₁;

R₆ is selected from the group consisting of SO₂R₁₁, SO₃R₁₁, C(O)OR₁₁, C(O)R₁₂ and C₀₋₄—P(O)R₁₃R₁₄;

R₇ is selected from the group consisting of hydroxy, C₁₋₈ alkyl and haloC₁₋₈ alkyl substituted with hydroxy;

R₈ and R₉ are each independently selected from the group consisting of hydrogen, deuterium, halogen, hydroxy, cyano, nitro, C₁₋₈ alkyl, C₂₋₈ alkenyl, C₂₋₈ alkynyl, C₃₋₈ cycloalkyl, C₁₋₈ alkoxy, C₃₋₈ cycloalkoxy, SO₂R₁₁, C(O)OR₁₁, C(O)R₁₂ and P(O)R₁₃R₁₄, wherein the alkyl and cycloalkyl are each optionally further substituted by one or more groups selected from the group consisting of halogen, hydroxy, C₁₋₈ alkyl, C₁₋₈ alkoxy, haloC₁₋₈ alkoxy, C₃₋₈ cycloalkyl and C₃₋₈ cycloalkoxy;

R₁₀ is selected from the group consisting of hydrogen, C₁₋₈ alkyl, C₃₋₈ cycloalkyl, halo C₁₋₈ alkyl, and C₁₋₈ alkoxy;

R₁₁ is selected from the group consisting of hydrogen, C₁₋₈ alkyl, C₃₋₈ cycloalkyl, halo C₁₋₈ alkyl, phenyl and p-methylphenyl;

R₁₂, R₁₃ and R₁₄ are each independently selected from the group consisting of hydroxy, C₁₋₈ alkyl, C₃₋₈ cycloalkyl, haloC₁₋₈ alkyl, C₁₋₈ alkoxy, amino, and C₁₋₈ alkyl disubstituted amino; and

n is 0 or 1.

In a preferred embodiment, in the cholic acid derivatives, the stereoisomer or the pharmaceutically acceptable salt thereof according to the present invention, the C₁₋₈ alkyl is preferably selected from C₁₋₆ alkyl, and more preferably selected from C₁₋₃ alkyl; the C₁₋₈ alkoxy is preferably selected from C₁₋₆ alkoxy, and more preferably selected from C₁₋₃ alkoxy; the C₃₋₈ cycloalkyl is preferably selected from C₃₋₆ cycloalkyl; the C₃₋₈ cycloalkoxy is preferably selected from C₃₋₆ cycloalkoxy; the C₂₋₈ alkenyl is preferably selected from C₂₋₄ alkenyl; and the C₂₋₈ alkynyl is preferably selected from C₂₋₄ alkynyl.

In a preferred embodiment, in the cholic acid derivatives, the stereoisomer or the pharmaceutically acceptable salt thereof according to the present invention, R₁ is selected from the group consisting of hydrogen, fluorine, methyl, trifluoromethyl, ethyl and isopropyl; and R₂, R₃, R₄, R₅, R₆, R₇, R₈, R₉, R₁₀, R₁₁, R₁₂, R₁₃, R₁₄, X₁, X₂, Y₁, Y₂, Z and n are as defined in the compound of formula (I).

In a preferred embodiment, in the cholic acid derivatives, the stereoisomer or the pharmaceutically acceptable salt thereof according to the present invention, R₂ is selected from the group consisting of hydrogen, methyl, ethyl and isopropyl; and R₁, R₃, R₄, R₅, R₆, R₇, R₈, R₉, R₁₀, R₁₁, R₁₂, R₁₃, R₁₄, X₁, X₂, Y₁, Y₂, Z and n are as defined in the compound of formula (I).

In a preferred embodiment, in the cholic acid derivatives, the stereoisomer or the pharmaceutically acceptable salt thereof according to the present invention, R₃ is selected from the group consisting of hydrogen, methyl, ethyl and isopropyl; and R₁, R₂, R₄, R₅, R₆, R₇, R₈, R₉, R₁₀, R₁₁, R₁₂, R₁₃, R₁₄, X₁, X₂, Y₁, Y₂, Z and n are as defined in the compound of formula (I).

In a more preferred embodiment, in the cholic acid derivatives, the stereoisomer or the pharmaceutically acceptable salt thereof according to the present invention, R₃ is selected from the group consisting of hydrogen and methyl; and R₁, R₂, R₄, R₅, R₆, R₇, R₈, R₉, R₁₀, R₁₁, R₁₂, R₁₃, R₁₄, X₁, X₂, Y₁, Y₂, Z and n are as defined in the compound of formula (I).

In the most preferred embodiment, the cholic acid derivatives, the stereoisomer or the pharmaceutically acceptable salt thereof according to the present invention is a compound of formula (II):

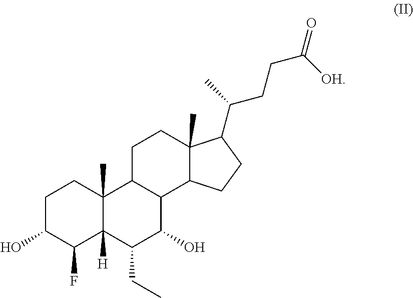 [SMILES: CC[C@@H]1[C@@H]2[C@@H](F)[C@H](O)CC[C@]2(C)C2CC[C@@]3(C)C(CCC3[C@H](C)CCC(=O)O)C2[C@@H]1O]

In a more preferred embodiment, in the cholic acid derivatives, the stereoisomer or the pharmaceutically acceptable salt thereof according to the present invention, R₁ is hydrogen; R₃ is selected from the group consisting of hydrogen and methyl; and R₂, R₄, R₅, R₆, R₇, R₈, R₉, R₁₀, R₁₁, R₁₂, R₁₃, R₁₄, X₁, X₂, Y₁, Y₂, Z and n are as defined in the compound of formula (I).

In the most preferred embodiment, the cholic acid derivatives, the stereoisomer or the pharmaceutically acceptable salt thereof according to the present invention is a compound of formula (III):

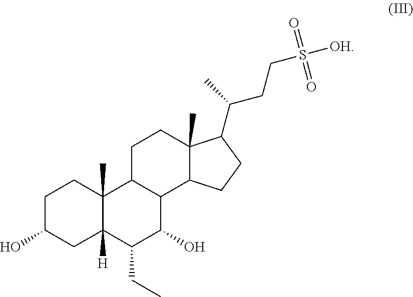 [SMILES: CC[C@H]1[C@@H](O)C2C3CCC([C@H](C)CCS(=O)(=O)O)[C@@]3(C)CCC2[C@@]2(C)CC[C@@H](O)C[C@@H]12]

In the most preferred embodiment, the cholic acid derivatives, the stereoisomer or the pharmaceutically acceptable salt thereof according to the present invention is a compound of formula (IV):

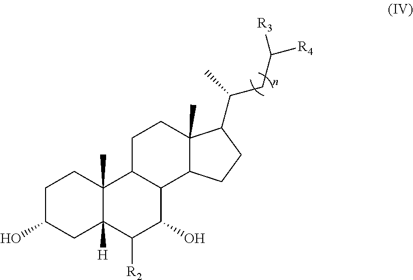

wherein R₄ is selected from the group consisting of:

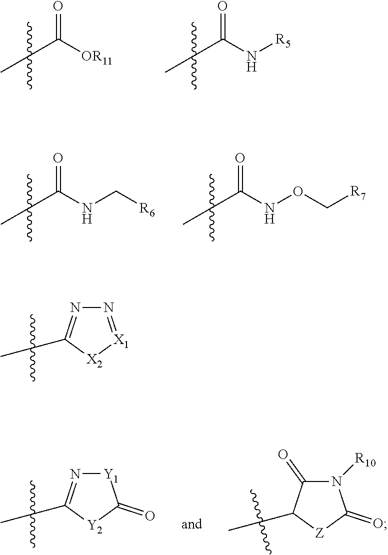

and R₂, R₃, R₅, R₆, R₇, R₈, R₉, R₁₀, R₁₁, R₁₂, R₁₃, R₁₄, X₁, X₂, Y₁, Y₂, Z and n are as defined in the compound of formula (I).

In a more preferred embodiment, in the cholic acid derivatives, the stereoisomer or the pharmaceutically acceptable salt thereof according to the present invention, when R₄ is selected from the group consisting of

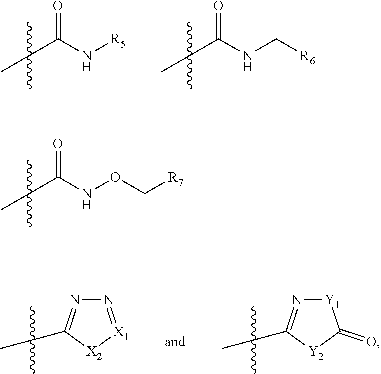

n is 1; when R₄ is

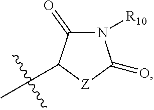

n is 0; and R₂, R₃, R₅, R₆, R₇, R₈, R₉, R₁₀, R₁₁, R₁₂, R₁₃, R₁₄, X₁, X₂, Y₁, Y₂ and Z are as defined in the compound of formula (I).

In the most preferred embodiment, the cholic acid derivatives, the stereoisomer or the pharmaceutically acceptable salt thereof according to the present invention is selected from the group consisting of:

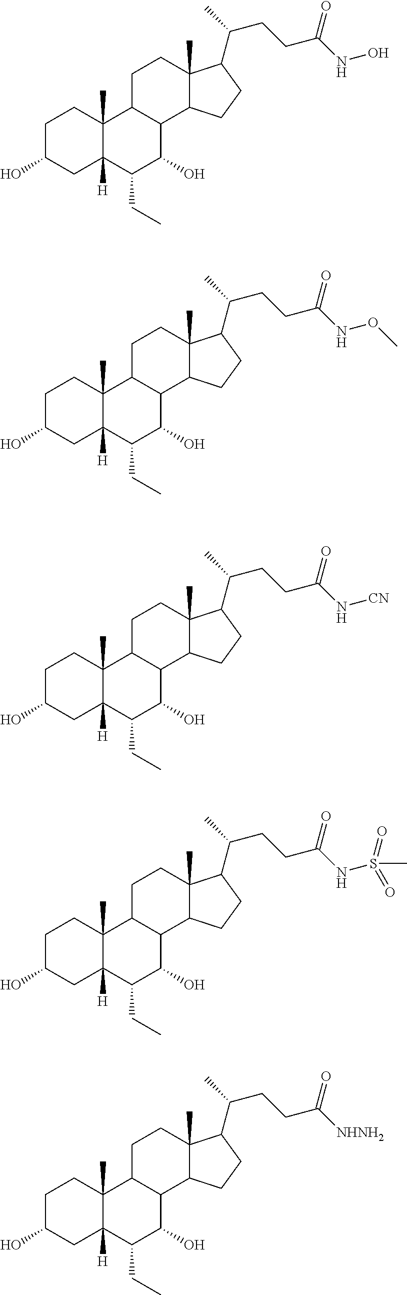 [SMILES: CC[C@H]1[C@@H](O)C2C3CCC([C@H](C)CCC(=O)NO)[C@@]3(C)CCC2[C@@]2(C)CC[C@@H](O)C[C@@H]12 ; CC[C@H]1[C@@H](O)C2C3CCC([C@H](C)CCC(=O)NOC)[C@@]3(C)CCC2[C@@]2(C)CC[C@@H](O)C[C@@H]12 ; CC[C@H]1[C@@H](O)C2C3CCC([C@H](C)CCC(=O)NC#N)[C@@]3(C)CCC2[C@@]2(C)CC[C@@H](O)C[C@@H]12 ; CC[C@H]1[C@@H](O)C2C3CCC([C@H](C)CCC(=O)NS(C)(=O)=O)[C@@]3(C)CCC2[C@@]2(C)CC[C@@H](O)C[C@@H]12 ; CC[C@H]1[C@@H](O)C2C3CCC([C@H](C)CCC(=O)NN)[C@@]3(C)CCC2[C@@]2(C)CC[C@@H](O)C[C@@H]12]

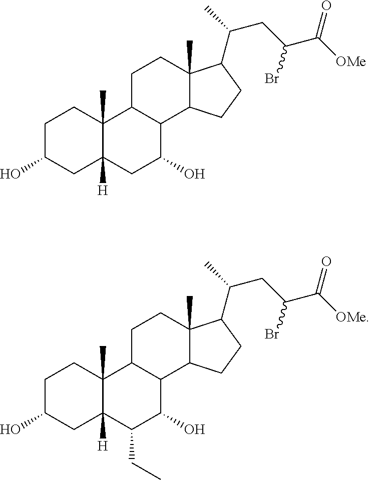 [SMILES: COC(=O)C(Br)C[C@@H](C)C1CCC2C3C(CC[C@@]21C)[C@@]1(C)CC[C@@H](O)C[C@H]1C[C@H]3O ; CC[C@H]1[C@@H](O)C2C3CCC([C@H](C)CC(Br)C(=O)OC)[C@@]3(C)CCC2[C@@]2(C)CC[C@@H](O)C[C@@H]12]

or selected from the group consisting of:

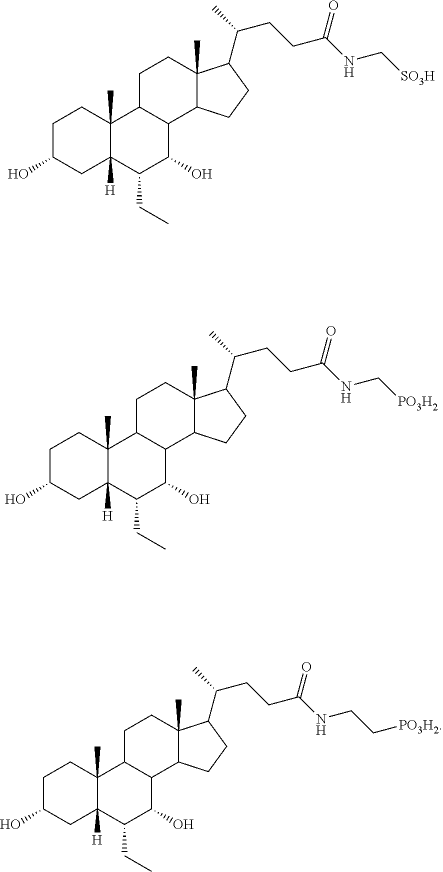

or selected from the compound of:

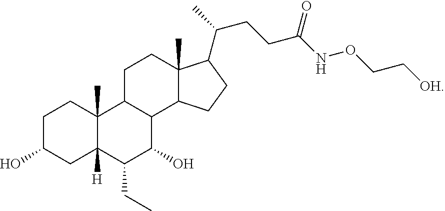 [SMILES: CC[C@H]1[C@@H](O)C2C3CCC([C@H](C)CCC(=O)NOCCO)[C@@]3(C)CCC2[C@@]2(C)CC[C@@H](O)C[C@@H]12]

In a more preferred embodiment, the cholic acid derivatives, the stereoisomer or the pharmaceutically acceptable salt thereof according to the present invention is a compound of formula (V):

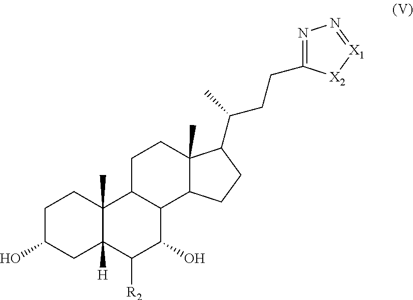

wherein, X₁, X₂, R₂, R₈, R₉ and R₁₀ are as defined in the compound of formula (I).

In the most preferred embodiment, the cholic acid derivatives, the stereoisomer or the pharmaceutically acceptable salt thereof according to the present invention is selected from the group consisting of:

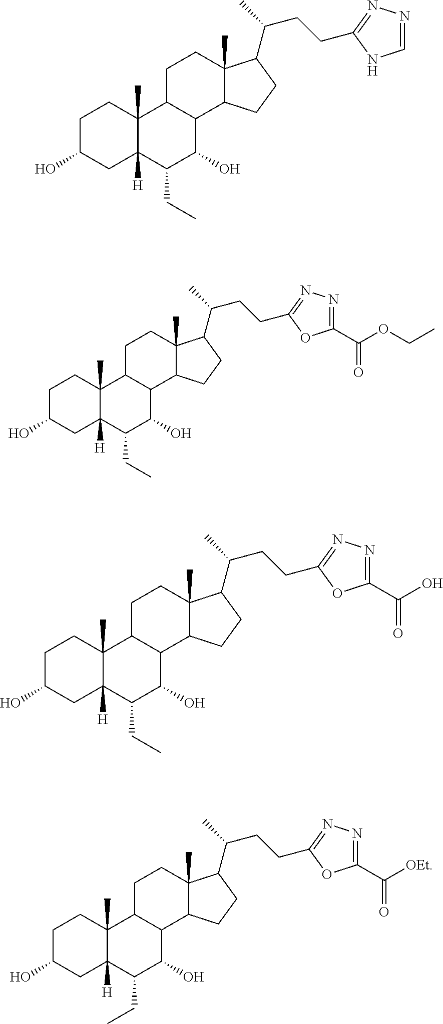 [SMILES: CC[C@H]1[C@@H](O)C2C3CCC([C@H](C)CCc4nnc[nH]4)[C@@]3(C)CCC2[C@@]2(C)CC[C@@H](O)C[C@@H]12 ; CCOC(=O)c1nnc(CC[C@@H](C)C2CCC3C4C(CC[C@@]32C)[C@@]2(C)CC[C@@H](O)C[C@H]2[C@@H](CC)[C@H]4O)o1 ; CC[C@H]1[C@@H](O)C2C3CCC([C@H](C)CCc4nnc(C(=O)O)o4)[C@@]3(C)CCC2[C@@]2(C)CC[C@@H](O)C[C@@H]12 ; CCOC(=O)c1nnc(CC[C@@H](C)C2CCC3C4C(CC[C@@]32C)[C@@]2(C)CC[C@@H](O)C[C@H]2[C@@H](CC)[C@H]4O)o1]

In a more preferred embodiment, the cholic acid derivatives, the stereoisomer or the pharmaceutically acceptable salt thereof according to the present invention is a compound of formula (VI):

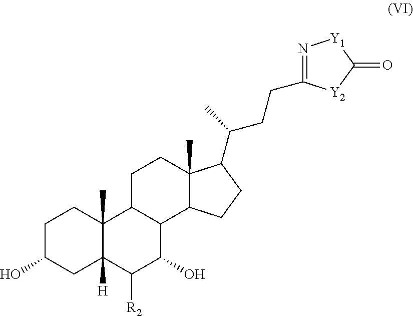

wherein, Y₁, Y₂, R₂, R₈, R₉, R₁₀, R₁₁, R₁₂, R₁₃ and R₁₄ are as defined in the compound of formula (I).

In the most preferred embodiment, the cholic acid derivatives, the stereoisomer or the pharmaceutically acceptable salt thereof according to the present invention is selected from the group consisting of:

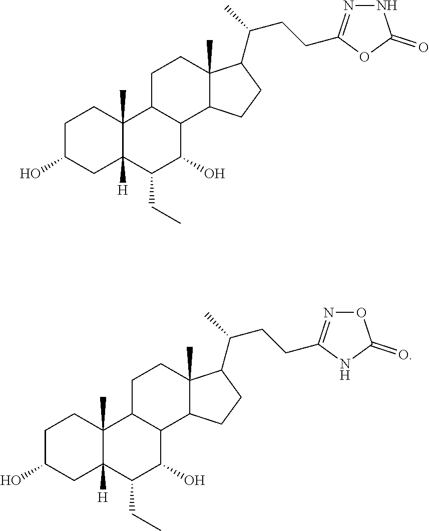 [SMILES: CC[C@H]1[C@@H](O)C2C3CCC([C@H](C)CCc4n[nH]c(=O)o4)[C@@]3(C)CCC2[C@@]2(C)CC[C@@H](O)C[C@@H]12 ; CC[C@H]1[C@@H](O)C2C3CCC([C@H](C)CCc4noc(=O)[nH]4)[C@@]3(C)CCC2[C@@]2(C)CC[C@@H](O)C[C@@H]12]

In a more preferred embodiment, the cholic acid derivatives, the stereoisomer or the pharmaceutically acceptable salt thereof according to the present invention is a compound of formula (VII):

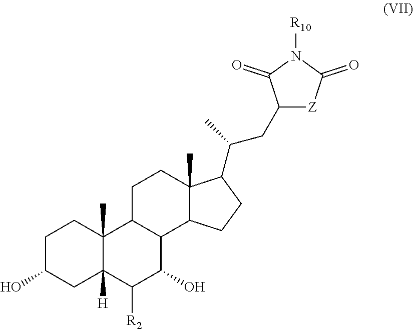

or a compound as defined below:

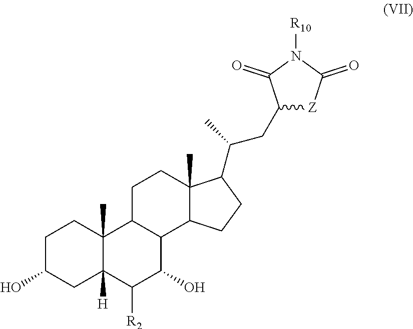

including two stereoisomers as defined below:

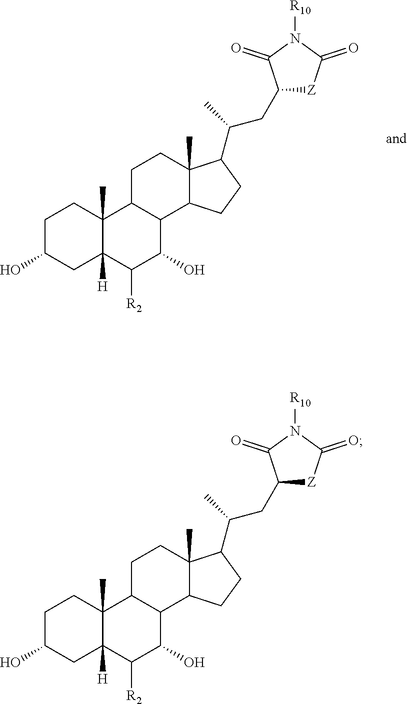

wherein, R₂, R₈, R₉, R₁₀, R₁₁, R₁₂, R₁₃, R₁₄ and Z are as defined in the compound of formula (I).

In the most preferred embodiment, the cholic acid derivatives, the stereoisomer or the pharmaceutically acceptable salt thereof according to the present invention is selected from the group consisting of:

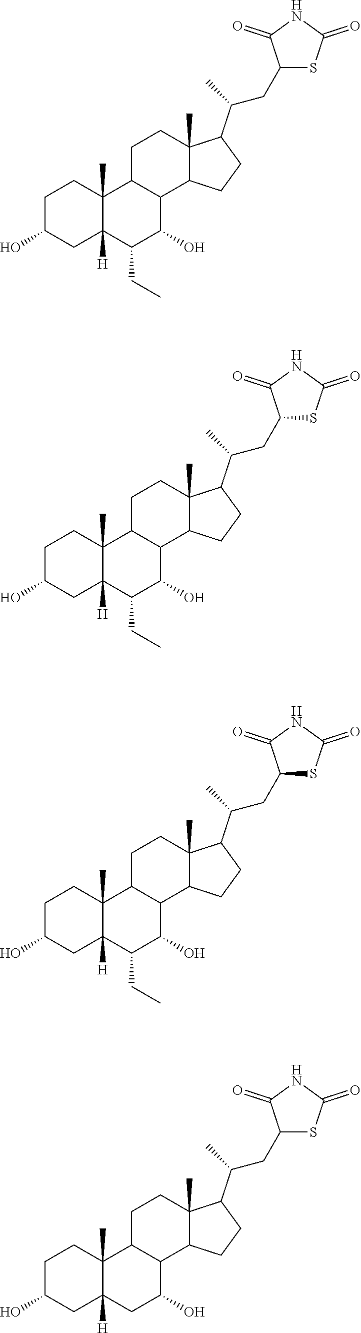 [SMILES: CC[C@H]1[C@@H](O)C2C3CCC([C@H](C)CC4SC(=O)NC4=O)[C@@]3(C)CCC2[C@@]2(C)CC[C@@H](O)C[C@@H]12 ; CC[C@H]1[C@@H](O)C2C3CCC([C@H](C)C[C@H]4SC(=O)NC4=O)[C@@]3(C)CCC2[C@@]2(C)CC[C@@H](O)C[C@@H]12 ; CC[C@H]1[C@@H](O)C2C3CCC([C@H](C)C[C@@H]4SC(=O)NC4=O)[C@@]3(C)CCC2[C@@]2(C)CC[C@@H](O)C[C@@H]12 ; C[C@H](CC1SC(=O)NC1=O)C1CCC2C3C(CC[C@@]21C)[C@@]1(C)CC[C@@H](O)C[C@H]1C[C@H]3O]

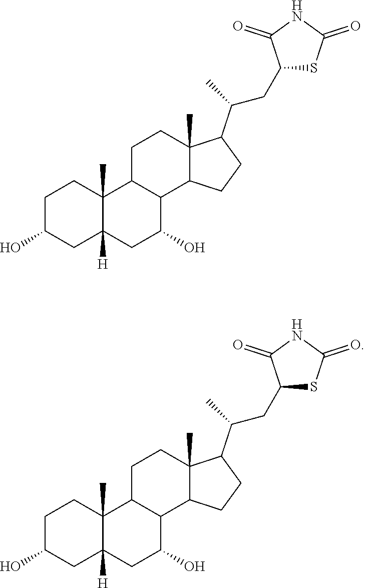 [SMILES: C[C@H](C[C@H]1SC(=O)NC1=O)C1CCC2C3C(CC[C@@]21C)[C@@]1(C)CC[C@@H](O)C[C@H]1C[C@H]3O ; C[C@H](C[C@@H]1SC(=O)NC1=O)C1CCC2C3C(CC[C@@]21C)[C@@]1(C)CC[C@@H](O)C[C@H]1C[C@H]3O]

In another aspect, the present invention provides the the cholic acid derivatives of formula (VIII), the stereoisomer or the pharmaceutically acceptable salt thereof, the compound of formula (VIII) is as defined below:

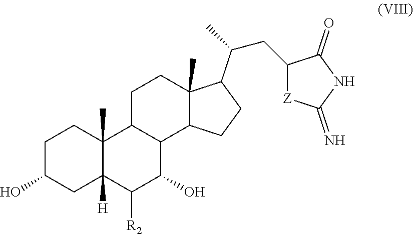

Z is selected from the group consisting of oxygen and sulfur atom;

R₂ is selected from the group consisting of hydrogen, fluorine, bromine, trifluoromethyl and C₁₋₈ alkyl.

In a more preferred embodiment, the cholic acid derivatives, the stereoisomer or the pharmaceutically acceptable salt thereof according to the present invention is selected from the group consisting of:

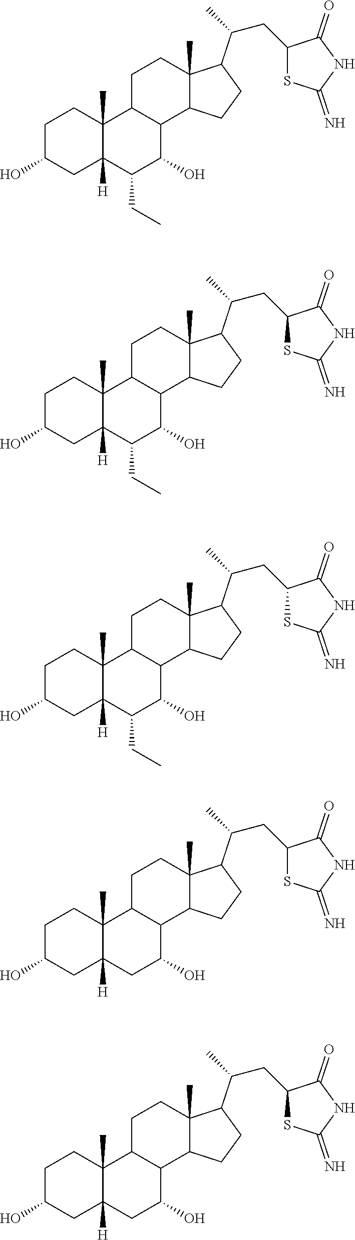 [SMILES: CC[C@H]1[C@@H](O)C2C3CCC([C@H](C)CC4SC(=N)NC4=O)[C@@]3(C)CCC2[C@@]2(C)CC[C@@H](O)C[C@@H]12 ; CC[C@H]1[C@@H](O)C2C3CCC([C@H](C)C[C@@H]4SC(=N)NC4=O)[C@@]3(C)CCC2[C@@]2(C)CC[C@@H](O)C[C@@H]12 ; CC[C@H]1[C@@H](O)C2C3CCC([C@H](C)C[C@H]4SC(=N)NC4=O)[C@@]3(C)CCC2[C@@]2(C)CC[C@@H](O)C[C@@H]12 ; C[C@H](CC1SC(=N)NC1=O)C1CCC2C3C(CC[C@@]21C)[C@@]1(C)CC[C@@H](O)C[C@H]1C[C@H]3O ; C[C@H](C[C@@H]1SC(=N)NC1=O)C1CCC2C3C(CC[C@@]21C)[C@@]1(C)CC[C@@H](O)C[C@H]1C[C@H]3O]

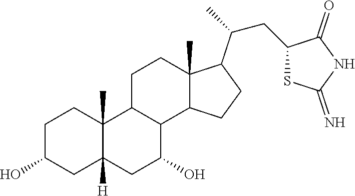 [SMILES: C[C@H](C[C@H]1SC(=N)NC1=O)C1CCC2C3C(CC[C@@]21C)[C@@]1(C)CC[C@@H](O)C[C@H]1C[C@H]3O]

In another aspect, the present invention provides a process for preparing the aforesaid cholic acid derivatives, the stereoisomer or the pharmaceutically acceptable salt thereof, comprising the steps of:

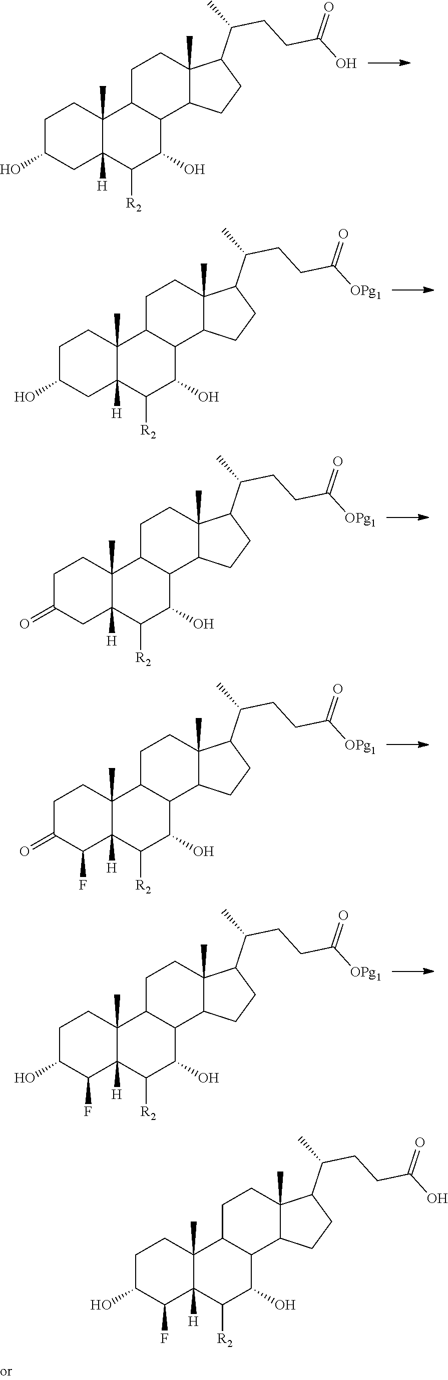

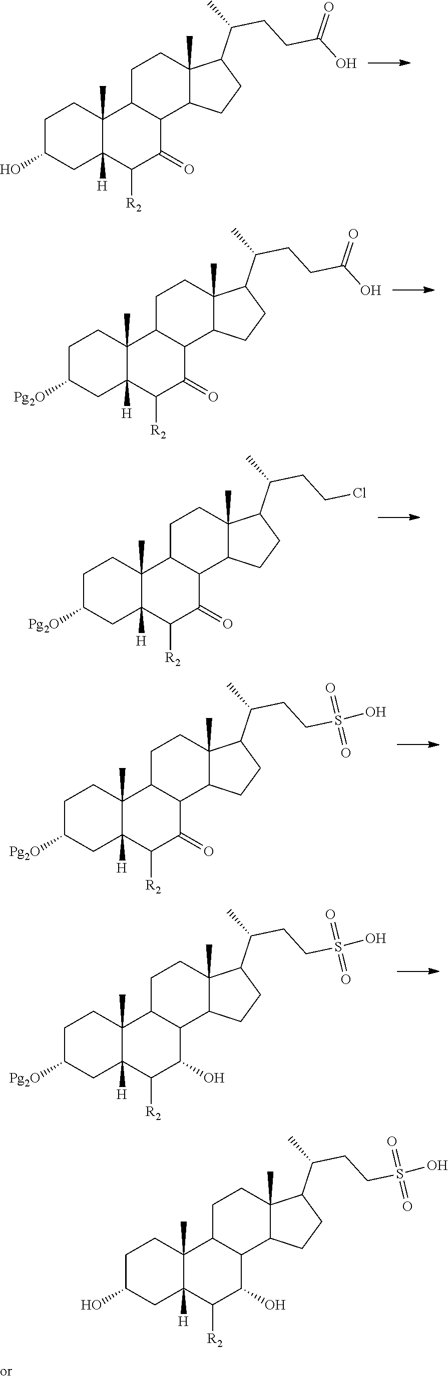

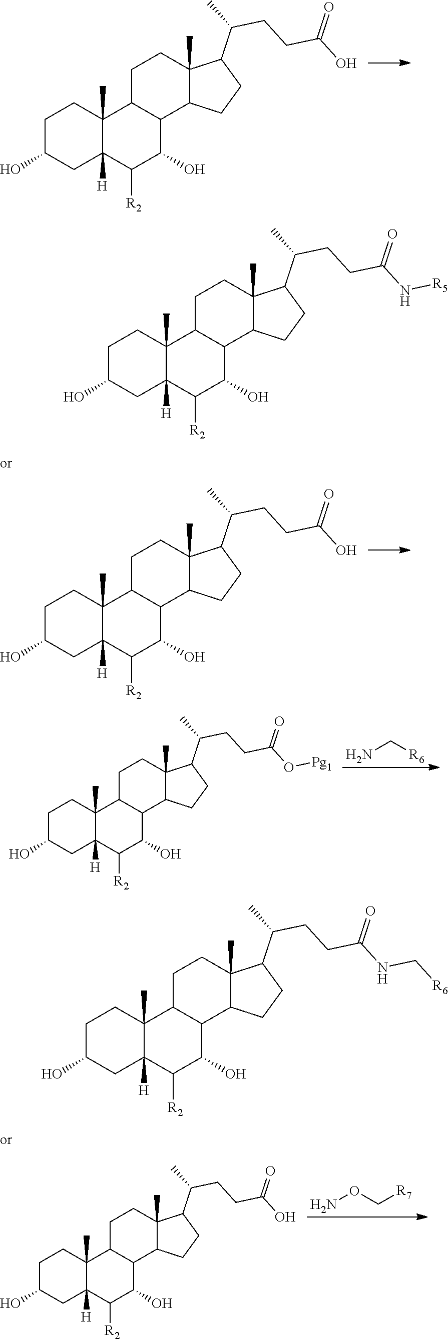

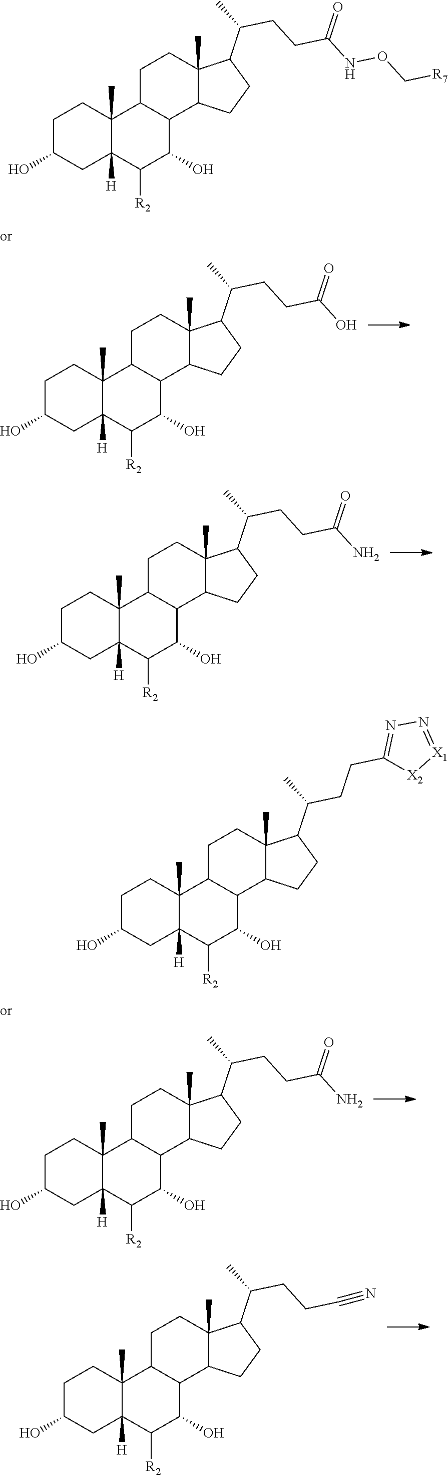

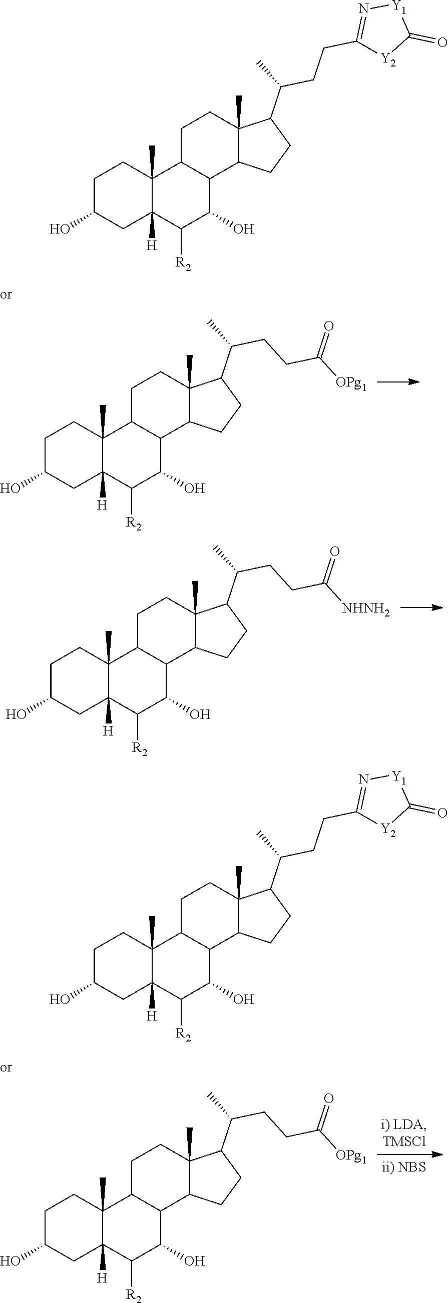

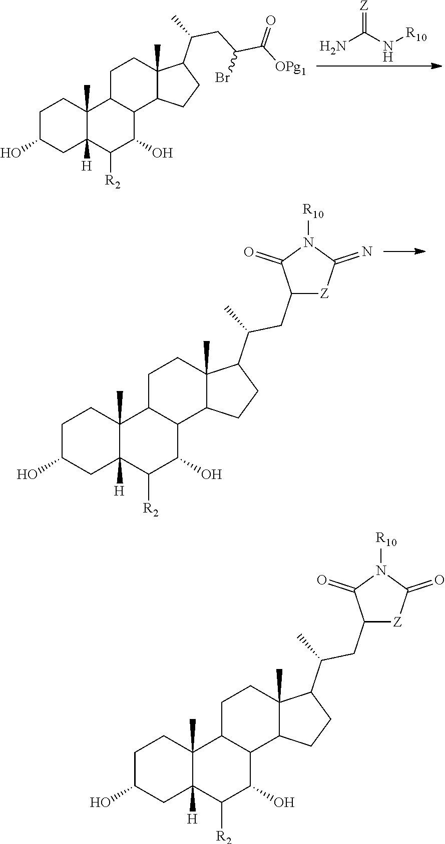

wherein, R₂, R₅, R₆, R₇, R₈, R₉, R₁₀, R₁₁, R₁₂, R₁₃, R₁₄, X₁, X₂, Y₁, Y₂ and Z are as defined in the compound of formula (I).

Pg₁ is a hydroxy protecting group, and is preferably selected from the group consisting of C₁₋₄ alkyl and benzyl; and Pg₂ is a hydroxy protecting group, and is preferably selected from the group consisting of C₁₋₄ alkyl and acetyl.

In another aspect, the present invention provides a pharmaceutical composition comprising a therapeutically effective amount of the aforesaid cholic acid derivatives, the stereoisomer or the pharmaceutically acceptable salt thereof and a pharmaceutically acceptable carrier.

In another aspect, the present invention provides a use of the aforesaid cholic acid derivatives, the stereoisomer or the pharmaceutically acceptable salt thereof, or the aforesaid pharmaceutical composition in the preparation of a medicament for preventing or treating FXR mediated diseases and conditions.

In a preferred embodiment, FXR mediated diseases and conditions are selected from the group consisting of cardiovascular disease, hypercholesterolemia, hyperlipidemia and chronic hepatitis disease, chronic liver disease, gastrointestinal disease, nephrosis, cerebrovascular disease, metabolic disease and cancer.

In a more preferred embodiment, the chronic liver disease is selected from the group consisting of primary cirrhosis (PBC), cerebrotendinous Xanthomatosis (CTX), primary sclerosing cholecystitis (PSC), cholestasis caused by drugs, intrahepatic cholestasis of pregnancy, parenteral nutrition associated cholestasis (PNAC), cholestasis of bacterial overgrowth or pyemia, autoimmune hepatitis, chronic viral hepatitis, alcoholic liver disease, non-alcoholic fatty liver disease (NAFLD), non-alcoholic steatohepatitis (NASH), graft versus host disease related to liver transplantation, regeneration of living donor liver transplantation, congenital hepatic fibrosis, choledocholithiasis, granulation liver disease, intrahepatic and extrahepatic malignant tumor, Sjogren syndrome, sarcoidosis, Wilson's disease, Gaucher's disease, hemochromatosis and α1-antitrypsin deficiency.

In a more preferred embodiment, the gastrointestinal disease is selected from the group consisting of inflammatory bowel disease (IBD) (incluing Crohn's disease and ulcerative enteritis), irritable bowel syndrome (IBS), bacterial overgrowth, nutrient malabsorption, colonitis after reflection and microscopic colitis.

In a more preferred embodiment, the nephrosis is selected from the group consisting of diabetic nephropathy, focal segmental glomerulosclerosis (FSGS), hypertensive nephropathy, chronic glomerulitis, chronic transplant glomerulopathy, chronic interstitial nephritis and polycystic kidney disease.

In a more preferred embodiment, the cardiovascular disease is selected from the group consisting of arteriosclerosis, arteriosclerosis, atherosclerosis, dyslipidemia, hypercholesterolemia and hypertriglyceridemia.

In a more preferred embodiment, the metabolic disease is selected from the group consisting of insulin resistance, type I diabetes, type II diabetes and obesity.

In a more preferred embodiment, the cerebrovascular disease is stroke.

In a more preferred embodiment, the cancer is selected from the group consisting of colorectal cancer and liver cancer.

In another aspect, the present invention provides a method for preventing and treating FXR mediated diseases and conditions, comprising administering a therapeutically effective amount of the aforesaid cholic acid derivatives, the stereoisomer or the pharmaceutically acceptable salt thereof, or the aforesaid pharmaceutical composition.

### DETAILED DESCRIPTION OF THE INVENTION

Detailed description: unless otherwise stated, the following terms which are used in the description and the claims have the following meanings.

“C₁₋₈ alkyl” refers to a straight chain or branched chain alkyl group having 1 to 8 carbon atoms. “Alkyl” refers to a saturated aliphatic hydrocarbon group, such as methyl, ethyl, n-propyl, isopropyl, n-butyl, isobutyl, t-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, n-heptyl, 2-methylhexyl, 3-methylhexyl, 4-methylhexyl, 5-methylhexyl, 2,3-dimethylpentyl, 2,4-dimethylpentyl, 2,2-dimethylpentyl, 3,3-dimethylpentyl, 2-ethylpentyl, 3-ethylpentyl, n-octyl, 2,3-dimethylhexyl, 2,4-dimethylhexyl, 2,5-dimethylhexyl, 2,2-dimethylhexyl, 3,3-dimethylhexyl, 4,4-dimethylhexyl, 2-ethylhexyl, 3-ethylhexyl, 4-ethylhexyl, 2-methyl-2-ethylpentyl, 2-methyl-3-ethylpentyl and various branched chain isomers thereof and the like.

“Cycloalkyl” refers to a saturated or partially unsaturated monocyclic or polycyclic hydrocarbon substituent. “C₃₋₈ cycloalkyl” refers to a cycloalkyl group having 3 to 8 carbon atoms, examples include:

Non-limiting examples of monocyclic cycloalkyl include cyclopropyl, cyclobutyl, cyclopentyl, cyclopentenyl, cyclohexyl, cyclohexenyl, cyclohexadienyl, cycloheptyl, cycloheptatrienyl, cyclooctyl and the like.
Polycyclic cycloalkyl includes a cycloalkyl having a spiro ring, fused ring and bridged ring. “Spiro cycloalkyl” refers to a polycyclic group with rings connected through one common carbon atom (called a spiro atom), wherein these rings can contain one or more double bonds, but none of the rings has a completely conjugated π electronic system. The spirocycloalkyl can be divided into mono-spiro cycloalkyl, di-spiro cycloalkyl or poly-spiro cycloalkyl according to the number of the spiro atoms shared between the rings. Non-limiting examples of spiro cycloalkyl include:

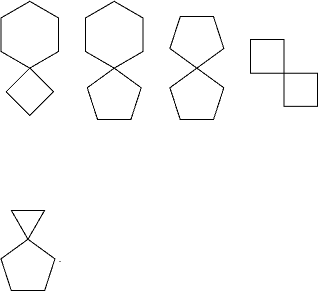

“Fused cycloalkyl” refers to an all-carbon polycyclic group in which each ring in the system shares an adjacent pair of carbon atoms with another ring, wherein one or more rings can contain one or more double bonds, but none of the rings has a completely conjugated π electronic system. According to the number of membered rings, the fused-cycloalkyl can be divided into bicyclic, tricyclic, tetracyclic and polycyclic fused cycloalkyl. Non-limiting examples of the fused cycloalkyl include:

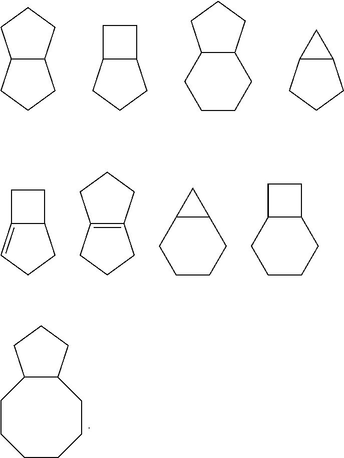

“Bridged cycloalkyl” refers to an all-carbon polycyclic group in which any two rings in the system share two disconnected carbon atoms, wherein these rings can contain one or more double bonds, but none of the rings has a completely conjugated π electronic system. According to the number of membered rings, the bridged cycloalkyl can be divided into bicyclic, tricyclic, tetracyclic and polycyclic bridged cycloalkyl. The non-limiting examples of the bridged cycloalkyl include:

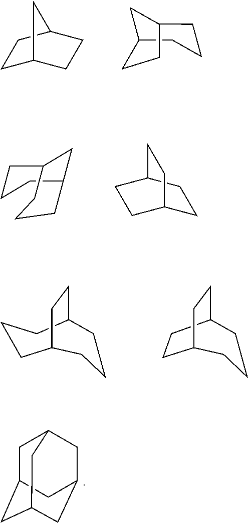

The cycloalkyl can be fused to the ring of aryl, heteroaryl or heterocyclyl, wherein the ring connected with the parent structure is the cycloalkyl, and non-limiting examples include indanyl, tetrahydronaphthyl, benzocycloheptylalkyl, and the like.

“Alkoxy” refers to an —O-(alkyl), wherein the alkyl is as defined above. “C₁₋₈ alkoxy” refers to an alkoxy having 1 to 8 carbons, and non-limiting examples include methoxy, ethoxy, propoxy, butoxy, and the like.

“Cycloalkoxy” refers to an —O-(unsubstituted cycloalkyl), wherein the cycloalkyl is as defined above. “C₃₋₈ cycloalkoxy” refers to a cycloalkoxy group having 3 to 8 carbons, and non-limiting examples include cyclopropoxy, cyclobutyloxy, cyclopentyloxy, cyclohexyloxy and the like.

“HaloC₁₋₈ alkyl” refers to a C₁₋₈ alkyl group, wherein hydrogens in the alkyl are substituted by fluorine, chlorine, bromine and iodine atoms, for example, difluoromethyl, dichloromethyl, dibromomethyl, trifluoromethyl, trichloromethyl, tribromomethyl, and the like.

“C(O)R₁₂” refers to a carbonyl group substituted with R₁₂.

“P(O)R₁₂R₁₃” refers to a phosphoryl substituted with R₁₂ and R₁₃, wherein R₁₂ and R₁₃ are optionally identical or different.

“PE” refers to petroleum ether.

“EA” refers to ethyl acetate.

“THF” refers to tetrahydrofuran.

“DCM” refers to dichloromethane.

“CDI” refers to N,N-carbonyldiimidazole

“DMF” refers to N,N-dimethylformamide.

“Dioxane” refers to 1,4-dioxane.

“DMAP” refers to 4-dimethylaminopyridine.

“DIPEA” refers to diisopropylethylamine.

“TMSCl ” refers to trimethylchlorosilane.

“LDA” refers to lithium diisopropylamide.

“Selectflour” refers to selective fluoride reagent.

“EDC.HCl” refers to 1-[3-(dimethylamino)propyl]-3-ethylcarbodiimide hydrochloride.

“TBTU” refers to O-benzotriazol-N,N,N′,N′-tetramethyluronium tetrafluoroborate.

“NBS” refers to N-bromosuccinimide.

“Optional” or “optionally” means that the subsequently described event or the circumstance can, but need not occur. Its meaning includes the instances in which the event or the circumstance does or does not occur. For example, “heterocyclyl optionally substituted by alkyl” means that the alkyl group can be, but need not be present. Its meaning includes the instances in which heterocyclyl is unsubstituted or substituted with alkyl.

“Substituted” means that one or more hydrogen atoms in a group are each independently substituted by the corresponding number of substituents. Obviously, the substituents are only positioned at their possible chemical positions, and the possible or impossible substitutions can be determined (through experiments or theory) by those skilled in the art without paying excessive efforts. For example, the combination of amino or hydroxy having a free hydrogen and carbon atoms having unsaturated bonds (such as olefinic) may be unstable.

“Pharmaceutical composition” refers to a mixture comprising one or more of the compounds described herein or the physiologically/pharmaceutically acceptable salts or prodrugs thereof and other chemical components, such as physiologically/pharmaceutically acceptable carriers and excipients. The purpose of the pharmaceutical composition is to facilitate administration of a compound to an organism, which will help absorption of the active ingredient, thereby realizing biological activity.

The following examples serve to illustrate the present invention in detail and completely, but these examples should not be considered as limiting the scope of the present invention, and the present invention is not limited to the examples.

The structures of compounds in the present invention can be identified by nuclear magnetic resonance (NMR) and/or liquid chromatography-mass spectrometry (LC-MS). The chemical shift of NMR is given in 10⁻⁶(ppm). NMR can be determined by a Bruker AVANCE-400 instrument, and the solvents for determination can be deuterated dimethylsulfoxide (DMSO-d6), deuterated methanol (CD₃OD) and deuterated chloroform (CDCl₃) with tetramethylsilane (TMS) as an internal standard.

Liquid chromatography-mass spectrometry (LC-MS) can be determined on an Agilent 1200 Infinity Series mass spectrometer. HPLC can be determined on an Agilent 1200DAD high pressure liquid chromatographic instrument (Sunfire C18 150×4.6 mm chromatographic column) and a Waters 2695-2996 high pressure liquid chromatographic instrument (Gimini C18 150×4.6 mm chromatographic column).

For thin-layer silica gel chromatography (TLC), Yantai Huanghai HSGF254 or Qingdao GF254 silica gel plate can be used. The dimension of the plates used in TLC can be 0.15 mm to 0.2 mm, and the dimension of the plates used in product purification can be 0.4 mm to 0.5 mm. Column chromatography generally uses Yantai Huanghai 200 to 300 mesh silica gel as carrier.

The starting materials used in the examples of the present invention are known and commercially available, or can be synthesized by adopting or according to methods known in the art.

Unless otherwise stated, all of the reactions of the present invention are carried out under continuous magnetic stirring in a dry nitrogen or argon atmosphere, and the solvent is dryied.

### EXAMPLE 1

### Preparation of (4R)-4-((3R, 4R, 5S, 6R, 7R, 10R, 13R)-6-ethyl-4-fluoro-3,7-dihydroxy-10,13-dimethylhexadecahydro-1H-cyclopenta[a] phenanthrene-17-yl)pentanoic acid

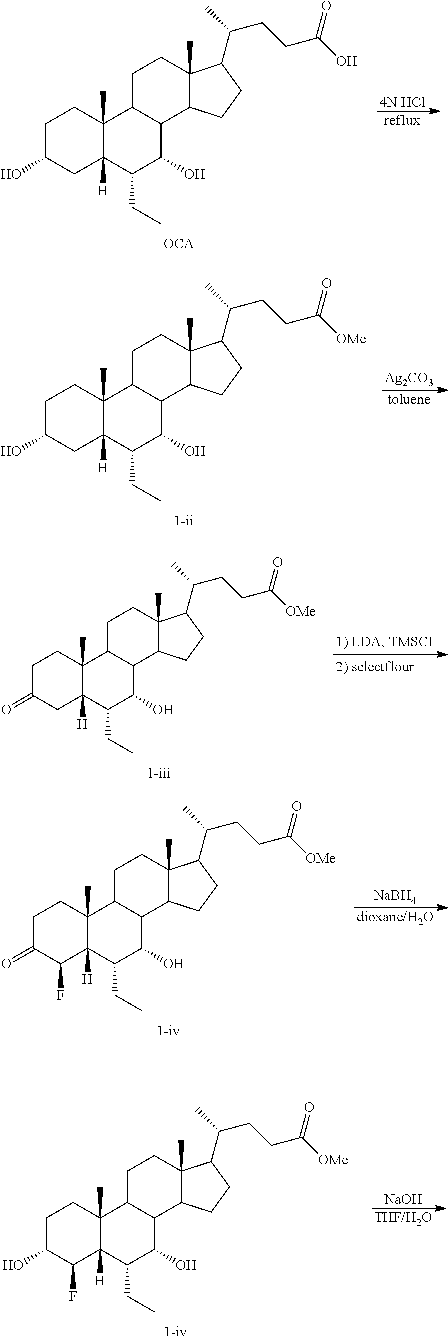

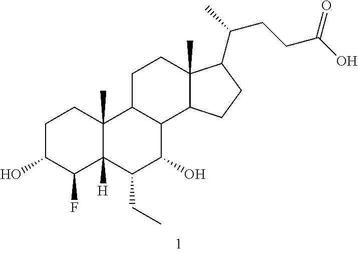 [SMILES: CC[C@@H]1[C@@H]2[C@@H](F)[C@H](O)CC[C@]2(C)C2CC[C@@]3(C)C(CCC3[C@H](C)CCC(=O)O)C2[C@@H]1O]

Step 1:

6α-Ethyl-chenodeoxycholic acid (OCA, 6.0 g, 14.2 mmol) was dissolved in a solution of 4N HCl in methanol and stirred at reflux for 2 hours. After TLC (PE:EA=1:2) showed completion of the reaction, methanol was removed by evaporation. The reaction solution was adjusted to basicity by adding saturated sodium bicarbonate, and extracted with 50 mL of ethyl acetate. The organic phase was dried over sodium sulfate, and concentrated under reduced pressure to obtain compound 1-ii (6.0 g, 98%).

Step 2:

Compound 1-ii (4.0 g, 9.2 mmol) was dissolved in 40 mL of toluene, and then added with silver carbonate (5.0 g, 18.4 mmol) and celatom (10 g). The reaction solution was stirred at reflux for 24 hours. After TLC (PE:EA=1:2) showed completion of the reaction, the reaction solution was filtered to remove celatom, and then washed with ethyl acetate several times. The organic phase was concentrated under reduced pressure and the residues were purified by column chromatography to obtain compound 1-iii (1.0 g, 25%).

Step 3:

Compound 1-iii (100 mg, 0.21 mmol) was dissolved in 2 mL of tetrahydrofuran, and then added with TMSCl (200 μL, 1.05 mmol) and LDA (90 μL, 0.42 mmol) at −78° C. The mixture was stirred for 3 hours, quenched with saturated sodium bicarbonate and extracted with ethyl acetate. The organic phase was dried over sodium sulfate and concentrated under reduced pressure to obtain an intermediate. The resulting product was dissolved in 2 mL of acetonitrile and stirred at room temperature for 1 hour after selectflour (105 mg, 0.29 mmol) was added. After TLC (PE:EA=5:1) showed completion of the reaction, 10 mL of water were added, and the reaction solution was extracted with ethyl acetate. The organic phase was concentrated under reduced pressure to obtain 100 mg of compound 1-iv, and the crude product was used directly in the next step.

Step 4:

Compound 1-iv (300 mg, 0.6 mmol) was dissolved in dioxane/H₂O (10 mL/0.5 mL), and sodium borohydride (49 mg, 1.3 mmol) was added in portions. The reaction mixture was stirred overnight at room temperature. One equivalent of sodium borohydride was added in the reaction mixture, and the stirring was continued for 4 hours. After TLC (PE:EA=5: 1) showed the disappearance of the starting material, the reaction was quenched with 10 mL of water, and extracted with ethyl acetate. The organic phase was dried over sodium sulfate and purified by column chromatography to obtain compound 1-v (40 mg, 15%).

Step 5:

Compound 1-v (40 mg, 0.088 mmol) was dissolved in 1 mL of tetrahydrofuran, and added with 1 mL of 1 N NaOH. The reaction solution was stirred at room temperature for 2 hours. After TLC (PE:EA=1:1) showed completion of the reaction, the reaction solution was adjusted to acidity by adding 1N HCl, and extracted with ethyl acetate. The organic phase was dried over sodium sulfate and purified by column chromatography to obtain compound 1 (5 mg, 12%).

¹H NMR (400 MHz, CDCl₃) δ 5.35-5.13 (m, 1H), 3.75 (s, 1H), 3.56-3.36 (m, 1H), 2.40-2.28 (m, 1H), 2.23-2.11 (m, 1H), 0.90 (s, 3H), 0.88-0.83 (m, 6H), 0.59 (s,3H).

¹³C NMR (101 MHz, CDCl₃) δ 178.50, 99.65 (d, J=168.8 Hz), 74.89 (d, J=20.0 Hz), 70.96, 55.78, 50.45, 49.37 (d, J=13.6 Hz), 42.77, 42.10, 40.33, 39.48, 39.00 (d, J=9.4 Hz), 35.39, 35.33, 34.44, 30.78, 29.69, 28.15, 26.02 (d, J=8.5 Hz), 24.28 (d, J=8.3 Hz), 23.60, 23.28, 20.75, 18.23, 12.48, 11.83.

¹⁹F NMR (376 MHz, CDCl₃) δ-188.73.

### EXAMPLE 2

### Preparation of (4R)-4-((3R,5S,6R,7R,10S,13R)-6-ethyl-3,7-dihydroxy-10,13-dimethylhexadecahydro-1H-cyclopenta[a]phenanthrene-17-yl)-N-hydroxypentanamide

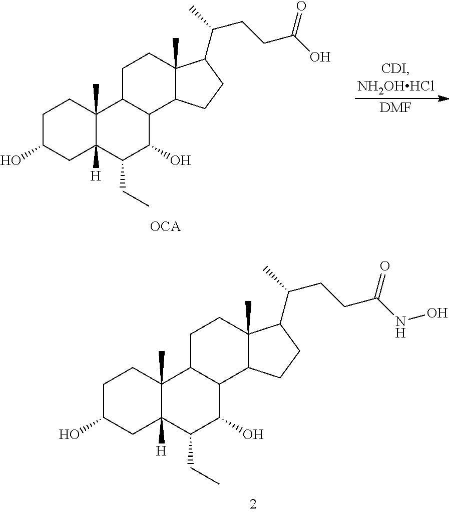

6α-Ethyl-chenodeoxycholic acid (OCA, 100 mg, 0.24 mmol) was dissolved in 2 ml of N,N-dimethylformamide and added with N,N-carbonyldiimidazole (77 mg, 0.48 mmol). The reaction mixture was stirred at room temperature for 0.5 hour. Then hydroxylamine hydrochloride (66 mg, 0.95 mmol) was added, and the reaction was continued at room temperature for 0.5 hour until completed. The reaction mixture was quenched with water, and extracted with ethyl acetate three times. The organic phase was dried over anhydrous sodium sulfate and filtered. The filtrate was concentrated under reduced pressure and the residues were purified by column chromatography to obtain compound compound 2 (16 mg, 15%).

¹H NMR (400 MHz, DMSO) δ 4.29 (d, J=4.2 Hz, 1H), 4.04 (d, J=5.0 Hz, 1H), 3.50 (brs, 1H), 3.20-3.10 (m, 2H), 2.05-1.61 (m), 1.60-0.76 (m), 0.60 (s, 3H).

¹³C NMR (101 MHz, DMSO) δ 169.99, 71.08, 68.89, 56.02, 50.60, 45.83, 42.50, 41.77, 36.01, 35.70, 35.42, 34.05, 33.14, 31.94, 30.93, 29.68, 29.44, 28.29, 23.57, 22.63, 20.89, 18.76, 12.22, 12.17.

### EXAMPLE 3

### Preparation of (4R)-N-cyano-4-((3R, 5S, 6R, 7R, 10S, 13R)-6-ethyl-3,7-dihydroxy-10,13-dimethylhexadecahydro-1H-cyclopenta[a] phenanthrene-17-yl)pentanamide

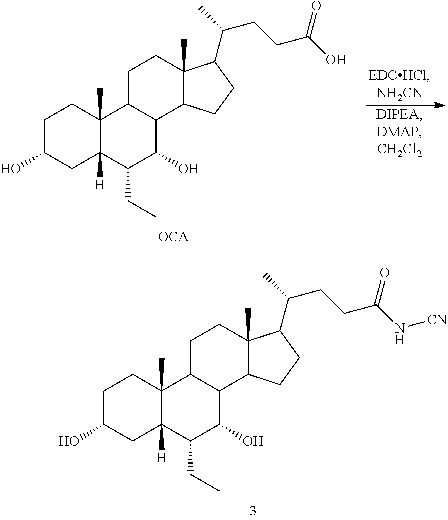 [SMILES: CC[C@H]1[C@@H](O)C2C3CCC([C@H](C)CCC(=O)O)[C@@]3(C)CCC2[C@@]2(C)CC[C@@H](O)C[C@@H]12 ; "DIPEA,DMAP,CH2Cl2" ; CC[C@H]1[C@@H](O)C2C3CCC([C@H](C)CCC(=O)NC#N)[C@@]3(C)CCC2[C@@]2(C)CC[C@@H](O)C[C@@H]12]

6α-Ethyl-chenodeoxycholic acid (OCA, 60 mg, 0.14 mmol), DMAP (21 mg, 0.17 mmol), EDC.HCl (38 mg, 0.20 mmol) and NH₂CN (12 mg, 0.28 mmol) were dissolved in 2 mL of dichloromethane, and added with DIPEA (50 μL, 0.28 mmol). The reaction mixture was stirred at room temperature overnight until completed. The reaction solution was diluted with dichloromethane, washed with dilute hydrochloric acid and saturated brine, successively. The organic phase was dried over anhydrous sodium sulfate and filtered. The filtrate was concentrated under reduced pressure and the residues were purified by column chromatography to obtain compound 3 (25 mg, 39%).

¹H NMR (400 MHz, DMSO) δ 4.28 (brs, 1H), 3.50 (brs, 1H), 3.13 (brs, 1H), 2.30 (dd, J=9.6, 5.1 Hz, 2H), 2.20 (dd, J=14.1, 5.8 Hz, 2H), 1.96-1.59 (m), 1.60-0.76 (m), 0.61 (s, 3H).

¹³C NMR (101 MHz, DMSO) δ 175.28, 71.08, 68.89, 55.92, 50.59, 45.82, 42.53, 41.76, 36.02, 35.70, 35.30, 34.04, 33.14, 32.40, 30.92, 29.44, 28.24, 23.57, 22.63, 20.89, 18.69, 12.18, 12.17.

### EXAMPLE 4

### Preparation of (4R)-4-((3R,5S,6R,7R,10S,13R)-6-ethyl-3,7-dihydroxy-10,13-dimethylhexadecahydro-1H-cyclopenta[a]phenanthrene-17-yl)-N-methoxypentanamide

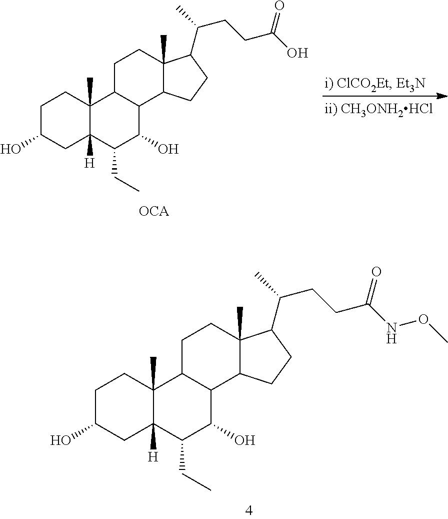 [SMILES: CC[C@H]1[C@@H](O)C2C3CCC([C@H](C)CCC(=O)O)[C@@]3(C)CCC2[C@@]2(C)CC[C@@H](O)C[C@@H]12 ; CC[C@H]1[C@@H](O)C2C3CCC([C@H](C)CCC(=O)NOC)[C@@]3(C)CCC2[C@@]2(C)CC[C@@H](O)C[C@@H]12]

6α-Ethyl-chenodeoxycholic acid (OCA, 42 mg, 0.1 mmol) was dissolved in 1 mL of N,N-dimethylformamide, and added with triethylamine (0.03 mL, 0.2 mmol). Then, ethyl chloroformate (0.01 mL, 0.11 mmol) was added in an ice bath and stirred for 0.5 hour. Methoxylamine hydrochloride (9 mg, 0.11 mmol) was dissolved in 1 mL of N,N-dimethylformamide, and then added with triethylamine (0.03 mL, 0.2 mmol). The solution was added dropwise to the reaction mixture above and the reaction temperature was gradually increased to room temperature. The reaction was completed after 3-4 hours. The reaction solution was washed with saturated brine, and extracted with ethyl acetate three times. The organic phases were combined and dried over anhydrous sodium sulfate, filtered and the filtrate was concentrated by column chromatography to obtain compound 4 (19.2 mg, 43%).

¹H NMR (400 MHz, CDCl₃) δ 3.69 (brs, 3H), 3.63 (brs, 1H), 3.40-3.27 (m, 1H), 1.96-1.02 (m), 1.00-0.75 (m), 0.59 (s, 3H).

¹³C NMR (101 MHz, MeOD) δ 171.86, 71.81, 69.79, 62.89, 55.87, 50.29, 45.57, 42.38, 41.78, 40.19, 39.66, 35.41, 35.26, 33.15, 33.05, 31.50, 29.88, 29.39, 27.89, 23.18, 22.39, 22.11, 20.60, 17.51, 10.87, 10.66.

### EXAMPLE 5

### Preparation of ((4R)-4-((3R,5S,6R,7R,10S,13R)-6-ethyl-3,7-dihydroxy-10,13-dimethylhexadecahydro-1H-cyclopenta[a]phenanthrene-17-yl)pentanoylamino) methanesulfonic acid

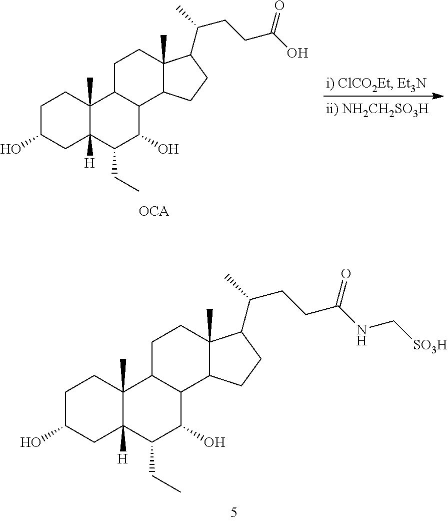 [SMILES: CC[C@H]1[C@@H](O)C2C3CCC([C@H](C)CCC(=O)O)[C@@]3(C)CCC2[C@@]2(C)CC[C@@H](O)C[C@@H]12 ; CC[C@H]1[C@@H](O)C2C3CCC([C@H](C)CCC(=O)NCS(=O)(=O)O)[C@@]3(C)CCC2[C@@]2(C)CC[C@@H](O)C[C@@H]12]

6α-Ethyl-chenodeoxycholic acid (OCA, 42 mg, 0.10 mmol) was dissolved in 1 mL of dry N,N-dimethylformamide, and added with triethylamine (28 μL, 0.20 mmol) and ethyl chloroformate (11 μL, 0.11 mmol), successively, in an ice-water bath. The reaction mixture was stirred at this temperature for 30 minutes. After the addition of aminomethanesulfonic acid (12 mg, 0.11 mmol), the reaction mixture was increased to room temperature and stirred for 5 hours. After completion of the reaction, the reaction solution was concentrated under reduced pressure, and the crude product was purified by column chromatography to obtain compound 5 (35 mg, 68%).

¹H NMR (400 MHz, MeOD) δ 4.42-4.25 (m, 2H), 3.67 (brs, 1H), 3.33 (brs, 1H), 2.48-2.31 (m, 1H), 2.28-2.13 (m, 1H), 2.11-1.70 (m), 1.68-0.84 (m), 0.71 (s, 3H).

¹³C NMR (101 MHz, MeOD) δ 175.42, 71.84, 69.86, 56.00, 55.50, 50.30, 45.57, 42.41, 41.75, 40.19, 39.69, 35.59, 35.45, 35.30, 33.15, 33.07, 32.71, 31.59, 29.93, 27.95, 23.25, 22.51, 22.12, 20.64, 17.68, 11.03, 10.77.

### EXAMPLE 6

### Preparation of (((4R)-4-((3R,5S,6R,7R,10S,13R)-6-ethyl-3,7-dihydroxy-10,13-dimethylhexadecahydro-1H-cyclopenta[a]phenanthrene-17-yl)pentanoylamino) methyl)phosphonic acid

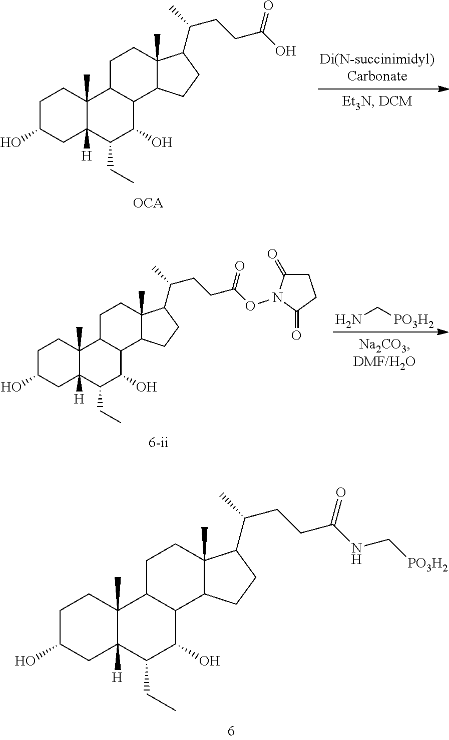

Step 1:

6α-Ethyl-chenodeoxycholic acid (OCA, 40 mg, 0.046 mmoL) was dissolved in dichloromethane, and added with di(N-succinimidyl) carbonate (20 mg, 0.051 mmol) and triethylamine (40 μL, 0.14 mmol), successively. The reaction mixture was stirred at room temperature for 4 hours. After TLC (PE:EA=1:2) showed the disappearance of starting material, 2 mL of water and 10 mL of dichloromethane were added. The organic phase was dried over sodium sulfate and concentrated under reduced pressure to obtain compound 6-ii.

Step 2:

The compound 6-ii was dissolved in DMF/H₂O (1 mL/1 mL), and added with sodium carbonate (12 mg, 0.11 mmol) and aminomethyl phosphoric acid. The reaction mixture was stirred at room temperature for 24 hours. After TLC (PE:EA=2:1) showed completion of the reaction, the reaction solution was adjusted to acidity by adding 1N HCl, and then 10 mL of ethyl acetate were added. The organic phase was washed with 2 mL of water three times, dried over sodium sulfate and concentrated under reduced pressure to obtain compound 6 (5.4 mg, 22%).

¹H NMR (400 MHz, MeOD) δ 3.55 (s, 1H), 3.48 (d, J=12.5 Hz, 2H), 3.23-3.19 (m, 1H), 0.88 (d, J=6.4 Hz, 3H), 0.85-0.74 (m, 6H), 0.60 (s, 3H).

¹³C NMR (101 MHz, MeOD) δ 175.22, 71.81, 69.81, 56.01, 50.28, 45.57, 42.36, 41.77, 40.18, 39.67, 36.78, 35.52, 35.39, 35.25, 33.15, 33.04, 32.48, 31.71, 29.87, 27.89, 23.18, 22.36, 22.10, 20.59, 17.52, 10.85, 10.63.

³¹P NMR (162 MHz, MeOD) δ 20.49 (s).

### EXAMPLE 7

### Preparation of (2-((4R)-4-((3R,5 S,6R,7R,10S,13R)-6-ethyl-3,7-dihydroxy-10,13-dimethylhexadecahydro-1H-cyclopenta[a]phenanthrene-17-yl)pentanoylamino)ethyl) phosphonic acid

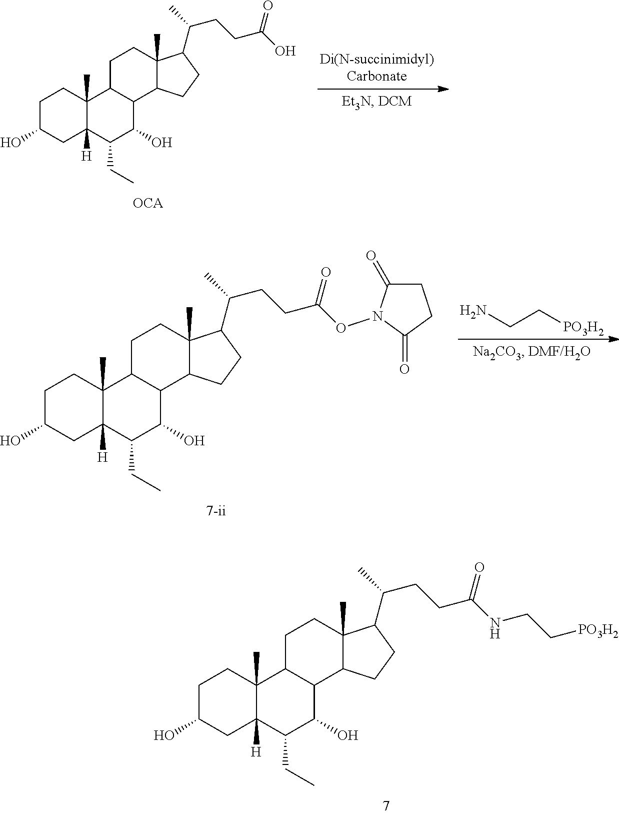

Step 1:

6α-Ethyl-chenodeoxycholic acid (OCA, 40 mg, 0.046 mmoL) was dissolved in dichloromethane, and added with di(N-succinimidyl) carbonate (20 mg, 0.051 mmol) and triethylamine (40 μL, 0.14 mmol), successively. The reaction mixture was stirred at room temperature for 4 hours. After TLC (PE:EA=1:2) showed the disappearance of starting material, 2 mL of water and 10 mL of dichloromethane were added. The organic phase was dried over sodium sulfate and concentrated under reduced pressure to obtain compound 7-ii.

Step 2:

The compound 7-ii was dissolved in DMF/H₂O (1 mL/1 mL), and added with sodium carbonate (12 mg, 0.11 mmol) and aminoethyl phosphoric acid . The reaction mixture was stirred at room temperature for 24 hours. After TLC (PE:EA=2:1) showed completion of the reaction, the reaction solution was adjusted to acidity by adding 1N HCl. Then, 10 mL of ethyl acetate were added, and the organic phase was washed with 2 mL of water three times, dried over sodium sulfate and concentrated under reduced pressure to obtain compound 7 (5.4 mg, 22%).

¹H NMR (400 MHz, MeOD) δ 3.62-3.52 (m, 1H), 3.38-3.27 (m, 1H), 3.24-3.20 (m, 2H), 0.92-0.84 (m, 3H), 0.84-0.76 (m, 6H), 0.60 (s, 3H).

¹³C NMR (101 MHz, MeOD) δ 175.31, 71.81, 69.81, 60.13, 55.96, 50.28, 45.58, 42.36, 41.78, 40.19, 39.66, 35.51, 35.39, 35.25, 33.15, 33.05, 31.79, 29.87, 29.32, 27.90, 23.17, 22.34, 22.10, 20.58, 17.52, 13.05, 10.84, 10.61.

³¹P NMR (162 MHz, MeOD) δ 25.30 (s).

### EXAMPLE 8

### Preparation of (4R)-4-((3R, 5S, 6R, 7R, 10S, 13R)-6-ethyl-3,7-dihydroxy-10,13-dimethylhexadecahydro-1H-cyclopenta[a]phenanthrene-17-yl)-N-(2-hydroxyethoxy)pentanamide

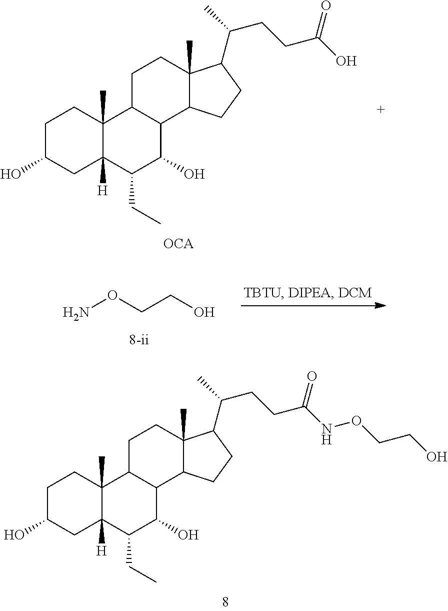 [SMILES: CC[C@H]1[C@@H](O)C2C3CCC([C@H](C)CCC(=O)O)[C@@]3(C)CCC2[C@@]2(C)CC[C@@H](O)C[C@@H]12 ; NOCCO ; CC[C@H]1[C@@H](O)C2C3CCC([C@H](C)CCC(=O)NOCCO)[C@@]3(C)CCC2[C@@]2(C)CC[C@@H](O)C[C@@H]12]

6α-Ethyl-chenodeoxycholic acid (OCA, 30 mg, 0.07 mmol) and compound 8-ii (6.5 mg, 0.085 mmoL) were dissolved in dichloromethane, and added with TBTU (40 mg, 0.11 mmoL) and DIPEA (30 μL, 0.21 mmoL), successively. The reaction solution was stirred at room temperature overnight. After TLC showed the disappearance of starting material, water was added, and the reaction solution was extracted with ethyl acetate. The organic phase was concentrated under reduced pressure and the residues were purified by column chromatography to obtain compound 8 (10 mg, 30%).

¹H NMR (400 MHz, MeOD) δ 3.95-3.85 (m, 1H), 3.74 (dt, J=16.4, 4.7 Hz, 2H), 3.67 (s, 1H), 3.35-3.32 (m, 1H), 1.00 (d, J=6.5 Hz, 3H), 0.95-0.89 (m, 6H), 0.72 (s, 3H).

¹³C NMR (101 MHz, MeOD) δ 172.75, 77.45, 71.80, 69.78, 59.02, 55.86, 50.29, 48.46, 45.57, 42.37, 41.77, 40.17, 39.65, 35.40, 35.25, 33.15, 33.04, 31.49, 29.87, 29.32, 27.90, 23.17, 22.36, 22.10, 20.59, 17.47, 10.85, 10.64.

### EXAMPLE 9

### Preparation of (3R)-3-((3R,5S,6R,7R,10S,13R)-6-ethyl-3,7-dihydroxy-10,13-dimethylhexadecahydro-1H-cyclopenta[a]phenanthrene-17-yl)butane-1-sulfonic acid

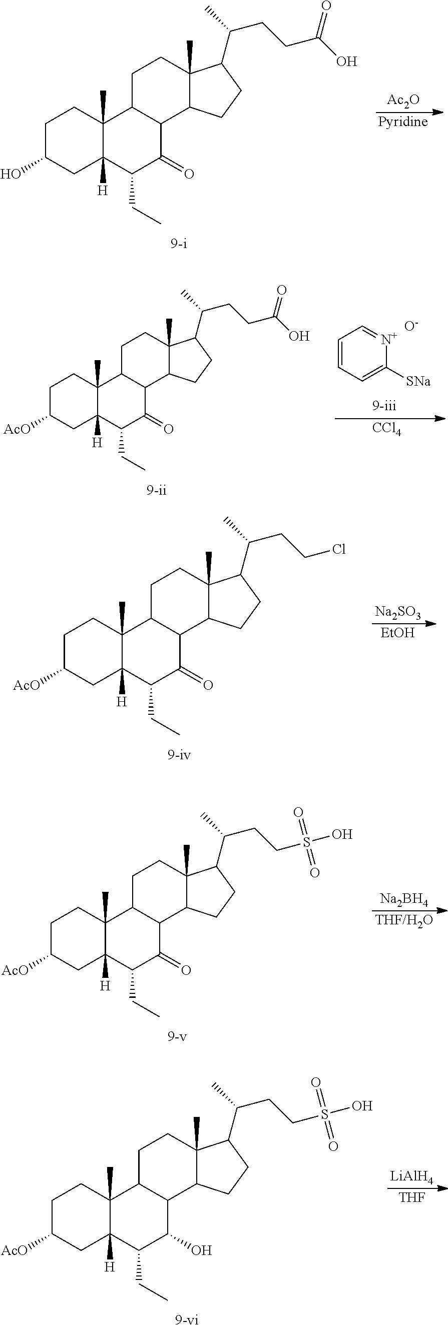

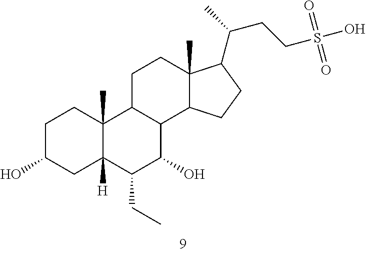 [SMILES: CC[C@H]1[C@@H](O)C2C3CCC([C@H](C)CCS(=O)(=O)O)[C@@]3(C)CCC2[C@@]2(C)CC[C@@H](O)C[C@@H]12]

Step 1:

Compound 9-i (1.5 g, 3.58 mmol) was dissolved in 20 mL of pyridine, and added with 15 mL of acetic anhydride. The reaction solution was stirred at room temperature overnight. After TLC (PE:EA=1:1) showed completion of the reaction, the solvent was removed by evaporation, and the residue was purified by column chromatography to obtain compound 9-ii (700 mg, 42%).

Step 2:

Compound 9-ii (100 mg, 0.21 mmol) was dissolved in 2 mL of dichloromethane, and added with a drop of DMF, then added with oxalyl chloride (18 671 L, 0.21 mmoL). The reaction solution was stirred at room temperature for 1 hour before TLC (PE:EA=5:1) showed completion of the reaction. The solvent was removed by evaporation, then the crude product was dissolved in 2 mL of acetonitrile, and added with compound 9-iii (38 mg, 0.25 mmol) and DMAP (1 mg, 0.05%). The mixture was stirred under nitrogen atmosphere for 2 hours. After TLC (PE:EA=5:1) showed completion of the reaction, compound 9-iv (110 mg, 90%) was obtained by column chromatography directly.

Step 3:

Compound 9-iv (300 mg, 0.66 mmol) was dissolved in 12 mL of ethanol, and added with 12 mL of sodium sulfite aqueous solution (20%). The reaction solution was heated to reflux for 24 hours. After TLC (PE:EA=10:1) showed completion of the reaction, 20 mL of water were added, and the solution was acidic. The reaction solution was extracted with ethyl acetate, dried over sodium sulfate and concentrated under reduced pressure to obtain compound 9-v (200 mg, 76%).

Step 4:

Compound 9-v (70 mg, 0.14 mmol) was dissolved in THF/H₂O (4 mL/0.4 mL) and stirred at room temperature for 2 hours after addition of 18 mg of sodium borohydride. The reaction was quenched with water, and extracted with ethyl acetate. The organic phase was concentrated under reduced pressure, and compound 9-vi (30 mg, 50%) was obtained by column chromatography.

Step 5:

Compound 9-vi (30 mg, 0.06 mmol) was dissolved in THF, and added with lithium aluminum hydride (10 mg, 0.27 mmol) under nitrogen atmosphere. The resulting solution was stirred at 70° C. overnight, then quenched with water and extracted with ethyl acetate. The organic phase was concentrated under reduced pressure, and compound 9 (1 mg, 3%) was obtained by column chromatography.

¹H NMR (400 MHz, CDCl₃) δ 3.70-3.51 (m, 3H), 3.42-3.27 (m, 1H), 0.91-0.87 (m, 3H), 0.85-0.79 (m, 6H), 0.61 (s, 3H).

¹³C NMR (101 MHz, CDCl₃) δ 72.36 70.93, 60.90, 56.38, 50.58, 45.25, 42.83, 41.23, 40.09, 39.67, 39.02, 35.56, 34.07, 33.29, 32.94, 30.70, 29.69, 28.40, 23.74, 23.15, 22.25, 20.77, 18.84, 11.77, 11.65.

### EXAMPLE 10

### Preparation of 5-((3R)-3-((3R,5S,6R,7R,10S,13R)-6-ethyl-3,7-dihydroxy-10,13-dimethylhexadecane-1H-cyclopenta[a]phenanthrene-17-yl)butyl)-1,3,4-oxadiazol-2 (3H) -one

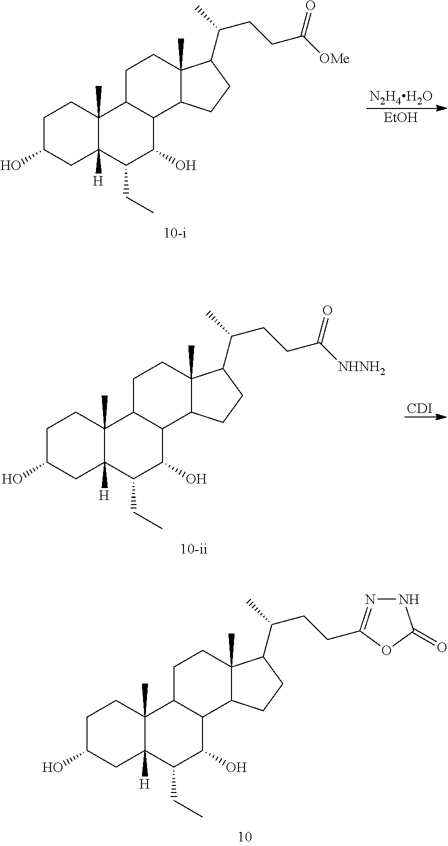 [SMILES: "EtOH" ; CC[C@H]1[C@@H](O)C2C3CCC([C@H](C)CCC(=O)OC)[C@@]3(C)CCC2[C@@]2(C)CC[C@@H](O)C[C@@H]12 ; "N2H4·H2O" ; CC[C@H]1[C@@H](O)C2C3CCC([C@H](C)CCC(=O)NN)[C@@]3(C)CCC2[C@@]2(C)CC[C@@H](O)C[C@@H]12 ; "CDI" ; CC[C@H]1[C@@H](O)C2C3CCC([C@H](C)CCc4n[nH]c(=O)o4)[C@@]3(C)CCC2[C@@]2(C)CC[C@@H](O)C[C@@H]12]

Step 1:

Methyl 6α-ethyl-chenodeoxycholicate 10-i (96 mg, 0.22 mmol) was dissolved in 2.5 mL of ethanol, and added with hydrazine hydrate (0.5 mL, 85%). The mixture was heated to reflux for 7 hours until the reaction was basically completed. After cooling to room temperature, the mixture was washed with saturated brine and extracted with ethyl acetate three times. The organic phases were combined and dried over anhydrous sodium sulfate, and then filtered. The filtrate was concentrated under reduced pressure to obtain compound 10-ii (87 mg, 91%).

Step 2:

The above crude product (43 mg, 0.10 mmol) was dissolved in 2 mL of dry tetrahydrofuran, and then added with N,N-carbonyldiimidazole (19 mg, 0.12 mmol). The mixture was stirred at room temperature overnight. After completion of the reaction, the mixture was washed with saturated brine and extracted with dichloromethane three times. The organic phases were combined and dried over anhydrous sodium sulfate, and then filtered. The filtrate was concentrated under reduced pressure to obtain crude product, which was purified by column chromatography to obtain compound 10 (20 mg, 43%).

¹H NMR (400 MHz, CDCl₃) δ 9.64 (s, 1H), 3.65 (brs, 1H), 3.44-3.25 (m, 1H), 2.54 (ddd, J=15.2, 10.5, 4.5 Hz, 1H), 2.38 (ddd, J=15.7, 9.9, 6.2 Hz, 1H), 2.12-1.01 (m), 1.00-0.75 (m), 0.59 (s, 3H).

¹³C NMR (101 MHz, CDCl₃) δ 158.71, 155.39, 72.31, 71.14, 55.62, 50.45, 45.17, 42.78, 41.23, 39.98, 39.60, 35.54, 35.51, 35.30, 33.91, 33.28, 31.38, 30.58, 28.22, 23.67, 23.39, 23.14, 22.23, 20.75, 18.25, 11.79, 11.65.

### EXAMPLE 11

### Preparation of (3R,5S,6R,7R,10S,13R)-17-((R)-4-(4H-1,2,4-triazol-3-yl) butan-2-yl)-6-ethyl-10,13-dimethylhexadecahydro-1H-cyclopenta[a] phenanthrene-3,7-diphenol

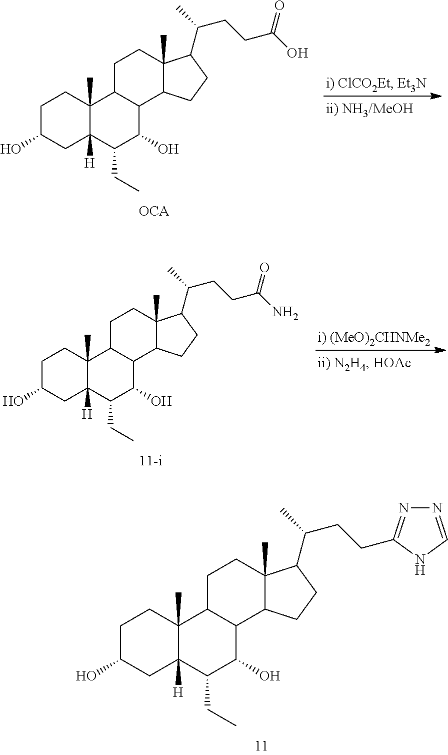 [SMILES: CC[C@H]1[C@@H](O)C2C3CCC([C@H](C)CCC(=O)O)[C@@]3(C)CCC2[C@@]2(C)CC[C@@H](O)C[C@@H]12 ; CC[C@H]1[C@@H](O)C2C3CCC([C@H](C)CCC(N)=O)[C@@]3(C)CCC2[C@@]2(C)CC[C@@H](O)C[C@@H]12 ; CC[C@H]1[C@@H](O)C2C3CCC([C@H](C)CCc4nnc[nH]4)[C@@]3(C)CCC2[C@@]2(C)CC[C@@H](O)C[C@@H]12]

Step 1:

6α-Ethyl-chenodeoxycholic acid (OCA, 42 mg, 0.10 mmol) was dissolved in 1 mL of dry N,N-dimethylformamide, and added with triethylamine (28 μL, 0.20 mmol) and ethyl chloroformate (11 μL, 0.11 mmol), successively, in an ice-water bath. The mixture was stirred for 30 minutes, and then added with 2.0 M solution of ammonia in methanol (0.10 mL, 0.20 mmol). The reaction mixture was warmed to room temperature and stirred for 2 hours until completed. The reaction solution was concentrated under reduced pressure to give a crude product that was used directly in the next step.

Step 2:

N,N-dimethylformamide dimethyl acetal (2 mL) was added to the above crude product, and the mixture was heated to reflux for 2 hours. After completion of the reaction, the mixture was cooled to room temperature and concentrated under reduced pressure. The resulting crude product was dissolved in 2 mL of acetic acid and then added with hydrazine hydrochloride (21 mg, 0.20 mmol). The reaction solution was heated to 90° C. and stirred for 90 minutes. The reaction solution was cooled to room temperature, quenched with water, and extracted with ethyl acetate. The organic phase was washed with saturated aqueous NaHCO₃, dried over anhydrous sodium sulfate, and filtered. The filtrate was concentrated under reduced pressure and the residues were purified by column chromatography to obtain compound 11 (21 mg, 47%).

¹H NMR (400 MHz, MeOD) δ 3.67 (brs, 1H), 2.94-2.80 (m, 1H), 2.73-2.66(m, 1H), 2.09-0.97 (m), 0.96-0.85 (m, 6H), 0.70 (s, 3H).

¹³C NMR (101 MHz, MeOD) δ 124.72, 71.80, 69.79, 55.87, 50.28, 45.57, 42.38, 41.77, 40.18, 39.66, 35.40, 35.25, 34.19, 33.15, 33.05, 29.88, 29.52, 27.92, 23.18, 22.37, 22.10, 20.59, 17.50, 10.83, 10.64.

### EXAMPLE 12

### Preparation of 3-((3R)-3-((3R, 5S, 6R, 7R, 10S, 13R)-6-ethyl-3,7-dihydroxy-10,13-dimethylhexadecahydro-1H-cyclopenta[a] phenanthrene-17-yl) butyl)-1,2,4-oxadiazol-5(4H)-one

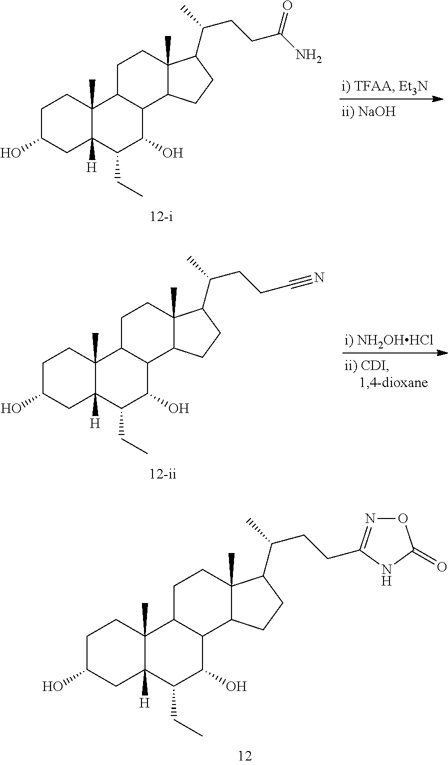 [SMILES: CC[C@H]1[C@@H](O)C2C3CCC([C@H](C)CCC(N)=O)[C@@]3(C)CCC2[C@@]2(C)CC[C@@H](O)C[C@@H]12 ; CC[C@H]1[C@@H](O)C2C3CCC([C@H](C)CCC#N)[C@@]3(C)CCC2[C@@]2(C)CC[C@@H](O)C[C@@H]12 ; CC[C@H]1[C@@H](O)C2C3CCC([C@H](C)CCc4noc(=O)[nH]4)[C@@]3(C)CCC2[C@@]2(C)CC[C@@H](O)C[C@@H]12]

Step 1:

6α-Ethyl-chenodeoxycholic amide 12-i (210 mg, 0.50 mmol) was dissolved in 5 ml of tetrahydrofuran, and then added with triethylamine (0.56 mL, 4.0 mmol) and trifluoroacetic anhydride (0.28 mL, 2.0 mmol), successively, in an ice-water bath. The mixture was stirred at room temperature for 3 hours. After completion of the reaction, the reaction solution was quenched with saturated aqueous NaHCO₃ solution and extracted with ethyl acetate twice. The organic phase was concentrated under reduced pressure and the residues were dissolved in 5 ml of tetrahydrofuran, and then 5 mL of 0.5 N aqueous solution of sodium hydroxide was added. The mixture was heated to 60° C. and stirred for 3 hours before extracting with ethyl acetate after completion. The organic phase was dried over anhydrous Na₂SO₄ and filtered. The filtrate was concentrated under reduced pressure and the residues were purified by column chromatography to obtain cyano compound 12-ii (167 mg, 83%).

Step 2:

Compound 12-ii (20 mg, 0.05 mmol), hydroxylamine hydrochloride (4 mg, 0.055 mmol) and NaHCO₃ (5 mg, 0.06 mmol) were added with 2 mL of methanol. The mixture was stirred at 120° C. under microwave for 15 hours. After about half of the reaction was completed, hydroxylamine hydrochloride (4 mg, 0.055 mmol) and NaHCO₃ (5 mg, 0.06 mmol) were added, and the reaction was continued at 120° C. under microwave for 12 hours until the reaction was basically completed. The reaction solution was concentrated under reduced pressure, extracted with ethyl acetate, and washed with saturated brine, dried over anhydrous Na₂SO₄, and then filtered. The filtrate was concentrated under reduced pressure. The resulting crude product was dissolved in 2 mL of 1,4-dioxane and added with CDI (12 mg, 0.075 mmol). The reaction mixture was heated to reflux for 2 hours until the starting material disappeared. Then, 3 mL of 0.1 N sodium hydroxide aqueous solution was added, and the mixture was heated to 50° C. and stirred for 1 hour. After cooling to room temperature, dilute hydrochloric acid was added to neutralize, and the mixture was extracted with ethyl acetate twice. The organic phase was washed with saturated brine and dried over anhydrous Na₂SO₄, and then filtered. The filtrate was concentrated under reduced pressure and the residues were purified by column chromatography to obtain compound 12 (15 mg, 65%).

¹H NMR (400 MHz, CDCl₃) δ 10.53 (s, 1H), 3.63 (brs, 1H), 3.46-3.30 (m, 1H), 2.62-2.48 (m, 1H), 2.47-2.35 (m, 1H), 2.11-0.71 (m), 0.60 (s, 3H). ¹³C NMR (101 MHz, CDCl₃) δ 161.06, 159.69, 72.46, 70.93, 55.64, 50.58, 45.14, 42.83, 41.19, 40.03, 39.65, 35.51, 35.34, 33.83, 33.30, 31.94, 31.44, 30.38, 30.21, 29.69, 28.30, 23.69, 23.12, 22.24, 22.09, 20.75, 18.26, 11.80, 11.66.

### EXAMPLE 13

### Preparation of 5-((3R)-3-((3R, 5S, 6R, 7R, 10S,13R)-6-ethyl-3,7-dihydroxy-10,13-dimethylhexadecahydro-1H-cyclopenta[a] phenanthrene-17-yl) butyl) -1,3,4-oxadiazole-2-carboxylic acid

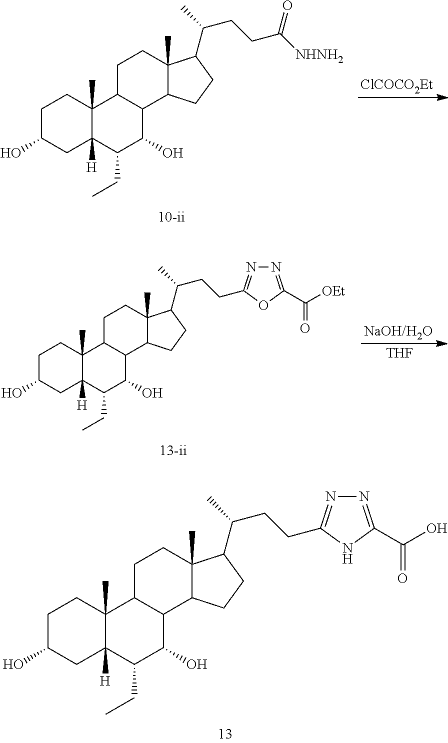 [SMILES: CC[C@H]1[C@@H](O)C2C3CCC([C@H](C)CCC(=O)NN)[C@@]3(C)CCC2[C@@]2(C)CC[C@@H](O)C[C@@H]12 ; CCOC(=O)c1nnc(CC[C@@H](C)C2CCC3C4C(CC[C@@]32C)[C@@]2(C)CC[C@@H](O)C[C@H]2[C@@H](CC)[C@H]4O)o1 ; CC[C@H]1[C@@H](O)C2C3CCC([C@H](C)CCc4nnc(C(=O)O)[nH]4)[C@@]3(C)CCC2[C@@]2(C)CC[C@@H](O)C[C@@H]12 ; "THF" ; "ClCOCO2Et"]

Step 1:

Methyl 6α-ethyl-chenodeoxycholicate 10-ii (43 mg, 0.10 mmol) was dissolved in 2 mL of chloroform, and then added with pyridine (12 μL, 0.15 mmol, 1.5 eq.) and oxalyl chloride monoethyl ester (13 μL, 0.12 mmol, 1.2 eq.), successively. The reaction mixture was heated to 80° C. and stirred for 2 hours until the reaction was basically completed. The reaction solution was concentrated under reduced pressure, and then washed with saturated brine and extracted with ethyl acetate three times. The organic phases were combined and dried over anhydrous sodium sulfate, filtered, and the filtrate was concentrated under reduced pressure. The residues were purified by column chromatography (CH₂Cl₂/MeOH 30:1) to obtain compound 13-ii (22 mg, 43%).

¹H NMR (400 MHz, CDCl₃) δ 4.31 (q, J=7.1 Hz, 2H), 3.63 (brs, 1H), 3.40-3.28 (m, 1H), 2.31 (ddd, J=14.9, 10.3, 4.8 Hz, 1H), 2.16 (ddd, J=14.7, 9.6, 4.7 Hz, 1H), 1.94-1.00 (m), 1.00-0.71 (m, H), 0.58 (s, 3H).

¹³C NMR (101 MHz, CDCl₃) δ 170.45, 158.61, 152.39, 72.34, 70.87, 63.61, 55.75, 50.54, 45.21, 42.77, 41.23, 40.06, 39.66, 35.51, 35.41, 33.90, 33.26, 31.34, 30.88, 30.59, 30.33, 28.23, 23.70, 23.15, 22.25, 20.77, 18.37, 13.97, 11.82, 11.67.

Step 2:

Compound 13-ii (16 mg, 0.031 mmol) was dissolved in 1 mL of tetrahydrofuran, and then added with 1 mL of sodium hydroxide aqueous solution (0.5 N). The reaction mixture was heated to 60° C. and stirred for 2 hours. After completion of the reaction, the reaction solution was adjusted to acidity by addition of diluted hydrochloric acid, and then extracted with ethyl acetate. Compound 13 (14 mg, 93%) was obtained by column chromatography (CH₂Cl₂/MeOH 5:1).

¹H NMR (400 MHz, MeOD) δ 3.56 (brs, 1H), 2.31-2.17 (m, 1H), 2.17-2.03 (m, 1H), 1.99-0.73 (m), 0.61 (s, 3H).

¹³C NMR (101 MHz, MeOD) δ 173.80, 160.17, 157.52, 71.81, 69.82, 55.97, 50.28, 45.57, 42.38, 41.77, 40.18, 39.67, 35.40, 35.25, 33.15, 33.05, 31.39, 30.36, 29.87, 27.87, 23.18, 22.37, 22.10, 20.59, 17.50, 10.88, 10.64.

### EXAMPLE 14

### Preparation of 5-((2R)-2-((3R, 5S, 6R, 7R, 10S, 13R)-6-ethyl-3,7-dihydroxy-10,13-dimethylhexadecahydro-1H-cyclopenta[a] phenanthrene-17-yl)propyl) thiazolidine-2,4-dione

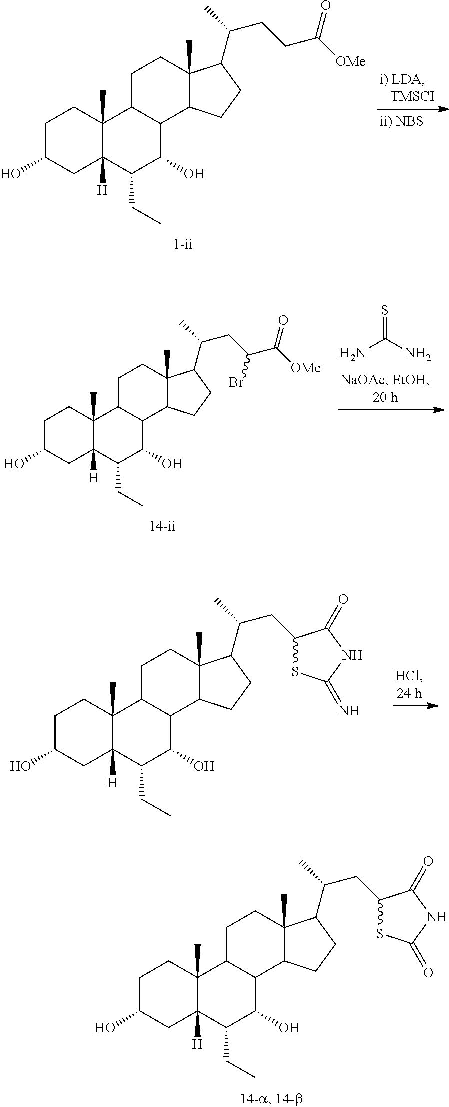

Step 1:

Methyl 6α-ethyl-chenodeoxycholicate 1-ii (845 mg, 1.94 mmoL) was dissolved in 10 mL of dry tetrahydrofuran, and the mixture was cooled to -72° C. in an ice-ethanol bath. LDA solution (4.9 mL, 9.72 mmol, 2 M) was added dropwise over 15 minutes and further stirred for 15 minutes before addition of trimethylchlorosilane (1.0 mL, 11.7 mmol) dropwise. The reaction mixture was stirred for 4 hours at low temperature. N-bromosuccinimide (692 mg, 3.89 mmol) was dissolved in 5 mL of tetrahydrofuran and then added dropwise to the reaction system. After completion of the addition, the reaction mixture was warmed to room temperature and stirred overnight. After TLC showed completion of the reaction, the reaction solution was quenched with 10 mL of water, and extracted with ethyl acetate three times. The organic phases were combined, washed with saturated brine, dried over anhydrous sodium sulfate, and filtered. The filtrate was concentrated under reduced pressure to obtain crude product 14-ii, which was used directly in the next step.

Step 2:

Compound 14-ii was dissolved in 20 ml of ethanol, and then added with thiourea (443 mg, 5.82 mmol) and sodium acetate (477 mg, 5.82 mmol). The mixture was heated to reflux and stirred overnight. After cooling to room temperature, 2N hydrochloric acid (20 mL) was added and the reaction was continued under reflux for 24 hours. The reaction solution was cooled, and concentrated under reduced pressure to remove ethanol. The residues were extracted with ethyl acetate three times. The organic phases were combined, dried over anhydrous sodium sulfate, filtered and the filtrate was concentrated under reduced pressure. The crude product was purified by column chromatography to obtain a mixture of diastereoisomers 14-α and 14-β (487 mg, 52%).

14-α: ¹H NMR (400 MHz, MeOD) δ 4.13 (dd, J=7.4, 5.8 Hz, 1H), 3.60 (brs, 1H), 3.31-3.20 (m, 1H), 2.30 (ddd, J=14.3, 5.6, 3.6 Hz, 1H), 1.96-1.02 (m), 1.02-0.75 (m, 12H), 0.61 (s, 3H).

¹³C NMR (101 MHz, MeOD) δ 177.04, 172.50, 71.95, 70.57, 56.15, 50.36, 49.34, 45.24, 42.90, 41.33, 40.17, 39.91, 39.56, 35.48, 35.44, 35.22, 33.32, 33.14, 30.17, 28.36, 23.53, 23.06, 22.10, 20.71, 18.76, 11.60, 11.46.

¹H NMR (400 MHz, CDCl₃) δ 4.31 (dd, J=11.3, 3.3 Hz, 1H), 3.70 (brs, 1H), 3.53-3.37 (m, 1H), 2.02-1.08 (m), 1.08-0.78 (m, 12H), 0.67 (s, 3H).

¹³C NMR (101 MHz, CDCl₃) δ 176.05, 171.36, 72.51, 71.02, 56.25, 50.49, 50.09, 45.10, 42.90, 41.24, 40.20, 39.97, 39.57, 35.54, 35.51, 35.45, 33.60, 33.08, 30.27, 28.39, 23.70, 23.09, 22.21, 20.72, 17.58, 11.81, 11.65.

### EXAMPLE 15

### Preparation of 5-((2R)-2-((3R,5 S,7R,10S,13R)-3,7-dihydroxy-10,13-dimethylhexadecahydro-1H-cyclopenta[a]phenanthrene-17-yl)propyl)thiazolidine-2,4-dione

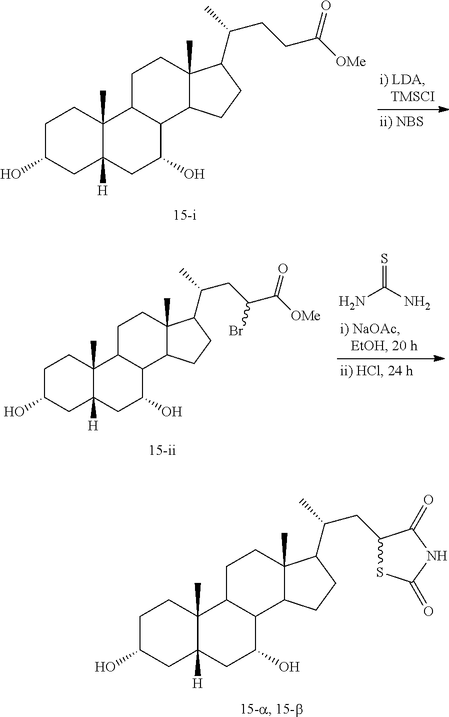

Step 1:

Methyl chenodeoxycholicate 15-i (348 mg, 0.86 mmol) was dissolved in 5 mL of dry tetrahydrofuran and the mixture was cooled to −72° C. in an ice-ethanol bath. LDA solution (2.1 mL, 4.28 mmol, 2M) was added dropwise in 15 minutes and further stirred for 15 minutes before addition of trimethylchlorosilane (0.44 mL, 5.14 mmol) dropwise. The reaction mixture was stirred for 4 hours at low temperature. N-bromosuccinimide (305 mg, 1.71 mmol) was dissolved in 5 mL of tetrahydrofuran and then added dropwise to the reaction system above. After completion of the addition, the reaction was warmed to room temperature and then stirred overnight. After TLC showed completion of the reaction, the reaction solution was quenched with 10 mL of water, and extracted with ethyl acetate three times. The organic phases were combined, washed with saturated brine, dried over anhydrous sodium sulfate, and filtered. The filtrate was concentrated under reduced pressure to obtain crude product 15-ii, which was used directly in the next step.

Step 2:

Compound 15-ii was dissolved in 10 mL of ethanol, and then added with thiourea (196 mg, 2.58 mmol) and sodium acetate (222 mg, 2.58 mmol). The mixture was heated to reflux and stirred for 6 hours. After cooling to room temperature, 2N hydrochloric acid (10 mL) was added and the reaction was continued under reflux overnight. The reaction solution was cooled to room temperature and concentrated under reduced pressure to remove ethanol, and then extracted with ethyl acetate three times. The organic phases were combined, dried over anhydrous sodium sulfate, filtered and the filtrate was concentrated under reduced pressure. The crude product was purified by column chromatography to obtain a mixture of diastereoisomers 15-α and 15-β (220 mg, 57%).

15-α: ¹H NMR (400 MHz, CDCl₃) δ 8.27 (s, 1H), 4.16 (dd, J=7.1, 6.1 Hz, 1H), 3.79 (d, J=2.3 Hz, 1H), 3.48-3.35 (m, 1H), 2.32 (ddd, J=14.3, 5.8, 3.6 Hz, 1H), 2.13 (dd, J=24.6, 12.8 Hz, 1H), 1.96-1.02 (m), 1.02-0.73 (m), 0.62 (s, 3H).

¹³C NMR (101 MHz, CDCl₃) δ 175.14, 170.32, 72.01, 68.54, 56.15, 50.45, 49.20, 42.99, 41.47, 40.25, 39.89, 39.64, 39.41, 35.33, 35.25, 35.05, 34.75, 32.87, 30.67, 28.42, 23.74, 22.75, 20.59, 18.89, 11.76.

15-β: ¹H NMR (400 MHz, CDCl₃) δ 9.87 (s, 1H), 4.23 (dd, J =11.5, 3.8 Hz, 1H), 3.79 (brs, 1H), 3.53-3.28 (m, 1H), 2.32-0.98 (m), 0.98-0.70 (m), 0.60 (s, 3H).

¹³C NMR (101 MHz, CDCl₃) δ 176.19, 171.57, 72.12, 68.62, 56.15, 50.33, 50.09, 42.81, 41.42, 40.26, 39.56, 39.31, 35.45, 35.33, 35.06, 34.78, 32.70, 30.37, 29.69, 28.41, 23.67, 22.74, 20.57, 17.58, 11.78.

### BIOLOGICAL EXPERIMENT EXAMPLE 1

### FXR Receptor Binding Activity Assay (Time-Resolved Fluorescence Detection Method TR-FRET Method)

To test the effect of the present compounds on FXR receptor binding, we selected the commercial FXR receptor binding activity assay kit to test the effect of the compounds on FXR and its binding protein fragments (LBD structural domain binds to SRC1 fragment) (kit supplier: Life Technology Invitrogen Catalog: PV4835; cisbio Catalog: 610SAXLA and 61GSTKLA). Specific method was described in the kit instructions. The operation was as follows:

1. Prepared 2× GST-labeled FXR-LBD/anti-GST Eu mixture to the required volume, the concentration of GST-FXR-LBD was 6 nM, Eu was 50 nL/well.

2. Prepared 2× biotin-labeled SRC1/streptomycin-allophycocyanin (SA-APC) mixture to the required volume, the concentration of SRC1 was 1000 nM, and streptomycin-allophycocyanin was 50 nL/well.

3. 2× GST-FXR/Eu and SRC1/SA-APC were mixed in a proportion of 1:1.

4. 20 μL of a mixture of GST-FXR/Eu and SRC1/SA-APC was added to a 384-well plate containing the compounds to be tested.

5. Centrifuged for 1 minute at 1000 rpm; incubated for 180 minutes at room temperature.

6. Time-resolved fluorescence detection: EnVision reader.

7.The analysis results:

(1) the value at 665 nm divided by the value at 615 nm;

(2) calculating the agonist rate

Agonist rate=(X−Min)/(Max−Min)*100%
X represents the 665/615 value for each concentration;
Min represents the 665/615 value without adding compound;
Max represents the 665/615 value of the reference compound.

The biochemical activity of the present compounds was determined by the above test, and the EC₅₀ values measured are shown in the table below.

| Example | EC₅₀ |
|---|---|
| Number | (uM) |
| 1 | 0.214 |
| 2 | 0.167 |
| 3 | 0.016 |
| 4 | 0.054 |
| 5 | 0.031 |
| 6 | 1.267 |
| 7 | 0.552 |
| 8 | 0.228 |
| 9 | 0.432 |
| 10 | 0.061 |
| 11 | 0.366 |
| 12 | 0.052 |
| 13-ii | 0.070 |
| 13 | 0.283 |
| 14-α | 0.013 |
| 14-β | 0.004 |
| 15-α | 0.143 |
| 15-β | 0.312 |

Conclusion: The example compounds of the present invention had a significant agonistic effect on FXR activity.

### BIOLOGICAL EXPERIMENT EXAMPLE 2

### FXR Reporter Gene Expression Test

To analyze the effect of testing compounds on FXR-regulated gene expression, we used Promega's dual luciferase assay system (Promega #E1980) to detect the effect of the compounds on the FXR reporter gene in HepG2 hepatoma cell line (ATCC #HB-8065), which was transiently transfected with the FXR Gal4 reporter gene fragment. Specific experimental methods are as follows:

1. HepG2 Cell Culture and Maintenance

Cells were digested and cells with appropriate density were grown in 10 mL complete medium and then cultivated for 24 hours under 37° C., 5% CO₂ humidity conditions.

2. Cell planting and transfection. Transfection reagent was Fugene HD (Promega#E231A)

(1) The transfection mixture was prepared according to the following table

| | |
|---|---|
| pBIND-FXR (ng/well) | 25 |
| pG5Luc (ng/well) | 25 |
| FuGENE HD (μL/well) | 0.15 |
| Serum-free medium (μL/well) | 1.85 |
| Total mixture (μL/well) | 2.5 |

(2) The mixture in the tube was vigorously mixed and incubated at room temperature for 15 minutes;

(3) The cells were digested in the culture dish and counted;

(4) The cell suspension was diluted to the desired volume of 600,000/mL (96-well plates 100 μL/well);

(5) The required volume of the transfection mixture was added to the cell suspension, followed by plating at 100 μL/well;

(6) Incubated for 24 hours under 37° C., 5% CO₂ humidity conditions.

3. Treatment of the Compound

(1) 10 mM of mother liquor of the compound was prepared and then serially diluted 3 times with DMSO;

(2) 10 μL of compound was added to 90 μL of complete medium (10×);

(3) 5 μL of the solution of the compound (21×) was added to each well;

(4) Incubated for 18 hours under 37° C., 5% CO₂ humidity conditions.

4. Dual-Luciferase Assay

The firefly luciferase and the renilla luciferase signal (Promega #E1980) were detected by Promega's dual-luciferase assay system.

5. Analysis of Results

(1) The detected values were given according to Firefly luciferase signal (F) divided by the renilla luciferase signal(R), F/R. This data processing eliminated the difference resulting from cell number and transfection efficiency.

(2) Calculating the agonist rate

Agonist rate=(X−Min)/(Max−Min)*100%
X represents the F/R value of each concentration;
Min represents the F/R value without adding compound;
Max represents the F/R value of the reference compound.

(3) EC₅₀ was calculated according to GraphPrism 5.0

The biochemical activity of the present compounds was determined by the above test, and the EC₅₀ values measured are shown in the table below.

| Example | EC₅₀ |
|---|---|
| Number | (uM) |
| 1 | 2.6 |
| 2 | 3.388 |
| 3 | 2.529 |
| 4 | 1.064 |
| 8 | 3.373 |
| 10 | 1.399 |
| 11 | 5.200 |
| 12 | 1.364 |
| 13 | 5.199 |
| 14-α | 0.063 |
| 14-β | 0.057 |
| 15-β | 3.37 |

Conclusion: The example compounds of the present invention had a significant agonistic effect on FXR activity.

### BIOLOGICAL EXPERIMENT EXAMPLE 3

### Pharmacokinetic Test in Rats

The pharmacokinetic study of the present invention was performed in rats. The pharmacokinetic behavior of the positive control INT-747 and a representative compound of the present invention, such as Example 14-β, was studied in rats, and their pharmacokinetic characteristics were evaluated.

1. Experimental Protocol

(1) Three male Sprague-Dawley (SD) rats were used, which were supplied by Sippr-BK laboratory animal Co. Ltd. Animal Production License No. SOCK (Shanghai) 2008-0016.

(2) Then, 10 mg of the sample was weighed and added with 10 mL of 0.5% CMC-Na aqueous solution, the mixture was fully shaken to make a 1 mg/ml uniform solution, and the remaining liquid samples were used for quantitative analysis.

(3) Three male SD rats were intragastrically administered at 10 mg/kg with a volume of 10 mL/kg after fasting for one night.

(4) About 0.05 mL of underjaw intravenous blood was collected before and 0.5, 1.0, 2.0, 4.0, 6.0, 8.0, 24.0, 30.0 hours after administration, and then placed in a EDTA-K2 test tube. The blood was centrifuged at 6000 rpm for 5 minutes to isolate the plasma, which was stored at −80° C. The rats were feed 4 hours after administration.

2. Analytical Methods

(1) Instruments and Equipment: API 4000 triple quadrupole mass spectrometer, Applied Biosystems, USA; Shimadzu LC-20AD high performance liquid chromatography system, Shimadzu, Japan.

(2) Condition of chromatography: The chromatographic column was Phenomenex gemiu 5 um C18 50×4.6 mm; mobile phase was acetonitrile: 0.1% TFA aqueous solution (gradient elution); flow rate (ml/min) was 0.8 ml/min.

(3) Condition of mass spectrometry:

| Potential of entrance | 10 | psi |
|---|---|---|
| Potential of collision cell export | 12 | psi |
| Curtain gas | 15 | psi |
| Collision Gas | medium | |
| Scanning mode | ESI(+), MRM | |
| Ionspray voltage | 5500 | V |
| Temperature | 550° | C. |
| Ion source gas 1 | 50 | psi |
| Ion source gas 2 | 50 | psi |

3. Pharmacokinetic Test Results:

(1) The results showed that the peak time of blood concentration tₘₐₓ was 0.5 hour, the peak concentration Cₘₐₓ was 4413 ng/mL, and the area under the blood concentration-time curve AUC₀₋ₜ was 9028 ng/mL·h after intragastric administration of 10 mg/kg of positive control INT-747.

(2) The results of the test showed that the peak time of blood concentration tₘₐₓ was 1 hour, the peak concentration Cₘₐₓ was 7577 ng/mL, and the area under the blood concentration-time curve AUC₀₋ₜ was 41796 ng/mL·h after the intragastric administration of 10 mg/kg of the compound of example 14-β

Pharmacokinetic tests in rats showed that the exposed quantity of the present compound of example 14-β in the tested animals was significantly better than that of the positive control INT-747.

1. A cholic acid derivative of formula (I), or a stereoisomer or a pharmaceutically acceptable salt thereof: 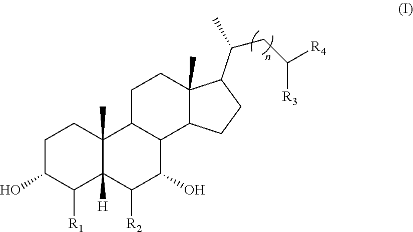
wherein
R₁, R₂ and R₃ are each selected from the group consisting of hydrogen, fluorine, bromine, trifluoromethyl and C₁₋₈ alkyl;
R₄ is selected from the group consisting of: 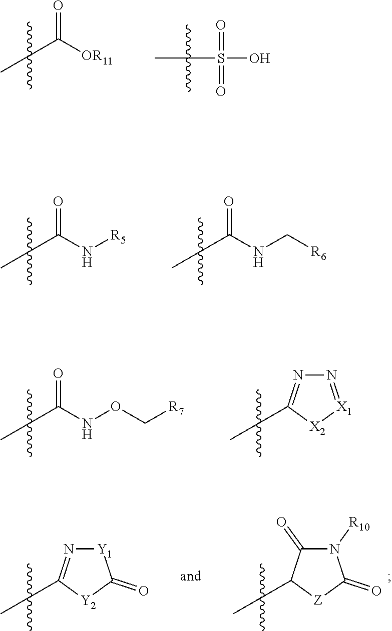
X₁ is selected from the group consisting of nitrogen and CR₈;
X₂ is selected from the group consisting of CR₈R₉, NR₁₀, oxygen and sulfur atom;
Y₁ and Y₂ are each independently selected from the group consisting of CR₈R₉, NR₁₀, oxygen and sulfur atom;
Z is selected from the group consisting of CR₈R₉, NR₁₀, oxygen and sulfur atom;
R₅ is selected from the group consisting of hydrogen, hydroxy, cyano, amino, C₁₋₈ alkoxy and SO₂R₁₁;
R₆ is selected from the group consisting of SO₂R₁₁, SO₃R₁₁, C(O)OR₁₁, C(O)R₁₂ and C₀₋₄—P(O)R₁₃R₁₄;
R₇ is selected from the group consisting of hydroxy, hydroxy substituted C₁₋₈ alkyl and haloC₁₋₈ alkyl;
R₈ and R₉ are each independently selected from the group consisting of hydrogen, deuterium, halogen, hydroxy, cyano, nitro, C₁₋₈ alkyl, C₂₋₈ alkenyl, C₂₋₈ alkynyl, C₃₋₈ cycloalkyl, C₁₋₈ alkoxy, C₃₋₈ cycloalkoxy, SO₂R₁₁, C(O)OR₁₁, C(O)R₁₂ and P(O)R₁₃R₁₄, wherein the alkyl and cycloalkyl are each optionally substituted by one or more groups selected from the group consisting of halogen, hydroxy, C₁₋₈ alkyl, C₁₋₈ alkoxy, haloC₁₋₈ alkoxy, C₃₋₈ cycloalkyl and C₃₋₈ cycloalkoxy;
R₁₀ is selected from the group consisting of hydrogen, C₁₋₈ alkyl, C₃₋₈ cycloalkyl, halo C₁₋₈ alkyl, and C₁₋₈ alkoxy;
R₁₁ is selected from the group consisting of hydrogen, C₁₋₈ alkyl, C₃₋₈ cycloalkyl, halo C₁₋₈ alkyl, phenyl and p-methylphenyl;
R₁₂, R₁₃ and R₁₄ are each independently selected from the group consisting of hydroxy, C₁₋₈ alkyl, C₃₋₈ cycloalkyl, halo C₁₋₈ alkyl, C₁₋₈ alkoxy, amino, and C₁₋₈ alkyl disubstituted amino; and
n is 0 or 1.

2. The cholic acid derivative, or the stereoisomer or pharmaceutically acceptable salt thereof according to claim 1, wherein C₁₋₈ alkyl is selected from the group consisting of C₁₋₆ alkyl, C₁₋₈ alkoxy is selected from the group consisting of C₁₋₆ alkoxy C₃₋₈ cycloalkyl is selected from the group consisting of C₃₋₆ cycloalkyl; C₃₋₈ cycloalkoxy is selected from the group consisting of C₃₋₆ cycloalkoxy; C₂₋₈ alkenyl is selected from the group consisting of C₂₋₄ alkenyl; and C₂₋₈ alkynyl is selected from the group consisting of C₂₋₄ alkynyl.

3. The cholic acid derivative, or the stereoisomer or pharmaceutically acceptable salt thereof according to claim 1, wherein R₃ is selected from the group consisting of hydrogen, methyl, ethyl and isopropyl; and R₁, R₂, R₄, R₅, R₆, R₇, R₈, R₉, R₁₀, R₁₁, R₁₂, R₁₃, R₁₄, X₁, X₂, Y₁, Y₂, Z and n are as defined in claim 1.

4. The cholic acid derivative, or the stereoisomer or pharmaceutically acceptable salt thereof according to claim 1, wherein R₃ is selected from the group consisting of hydrogen and methyl; and R₁, R₂, R₄, R₅, R₆, R₇, R₈, R₉, R₁₀, R₁₁, R₁₂, R₁₃, R₁₄, X₁, X₂, Y₁, Y₂, Z and n are as defined in claim 1.

5. The cholic acid derivative, or the stereoisomer or pharmaceutically acceptable salt thereof according to claim 1, wherein the compound is a compound of formula (II):

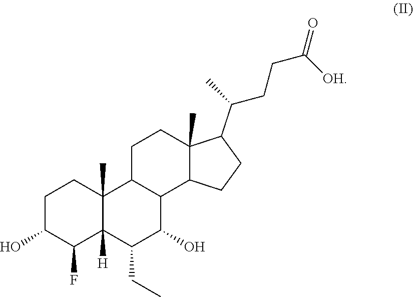 [SMILES: CC[C@@H]1[C@@H]2[C@@H](F)[C@H](O)CC[C@]2(C)C2CC[C@@]3(C)C(CCC3[C@H](C)CCC(=O)O)C2[C@@H]1O]

6. The cholic acid derivative, or the stereoisomer or pharmaceutically acceptable salt thereof according to claim 1, wherein R₁ is hydrogen; R₃ is selected from the group consisting of hydrogen and methyl; and R₂, R₄, R₅, R₆, R₇, R₈, R₉, R₁₀, R₁₁, R₁₂, R₁₃, R₁₄, X₁, X₂, Y₁, Y₂, Z and n are as defined in claim 1.

7. The cholic acid derivative, or the stereoisomer or pharmaceutically acceptable salt thereof according to claim 1, wherein the compound is a compound of formula (III):

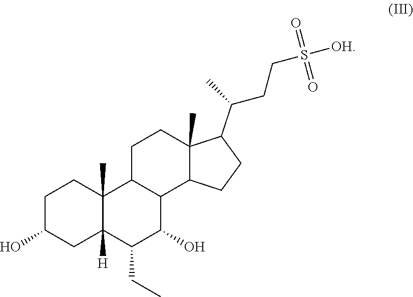 [SMILES: CC[C@H]1[C@@H](O)C2C3CCC([C@H](C)CCS(=O)(=O)O)[C@@]3(C)CCC2[C@@]2(C)CC[C@@H](O)C[C@@H]12]

8. The cholic acid derivative, or the stereoisomer or pharmaceutically acceptable salt thereof according to claim 1, wherein the compound is a compound of formula (IV) 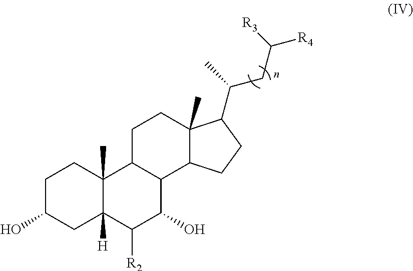
wherein R₄ is selected from the group consisting of: 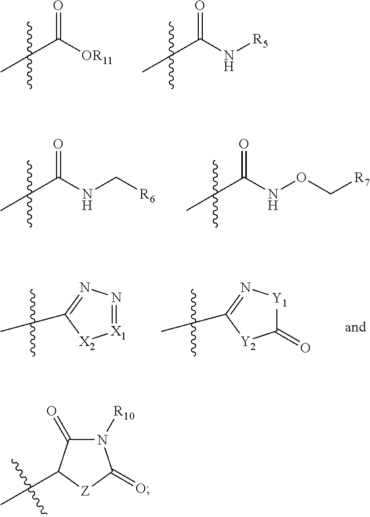
and R₂, R₃, R₅, R₆, R₇, R₈, R₉, R₁₀, R₁₁, R₁₂, R₁₃, R₁₄, X₁, X₂, Y₁, Y₂, Z and n are as defined in claim 1.

9. The cholic acid derivative, or the stereoisomer or pharmaceutically acceptable salt thereof according to claim 8, wherein when R₄ is selected from the group consisting of 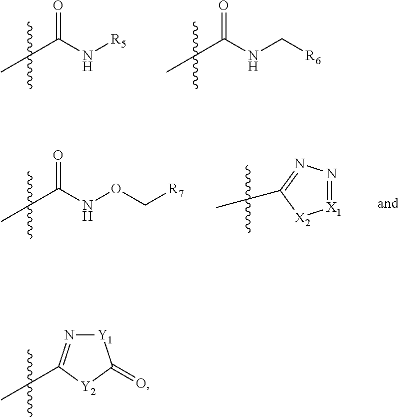
then n is 1;
where R₄ is 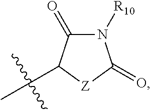
then n is 0; and
R₂, R₃, R₅, R₆, R₇, R₈, R₉, R₁₀, R₁₁, R₁₂, R₁₃, R₁₄, X₁, X₂, Y₁, Y₂ and Z are as defined in claim 8.

10. The cholic acid derivative, or the stereoisomer or pharmaceutically acceptable salt thereof according to claim 8, wherein the compound is selected from the group consisting of:

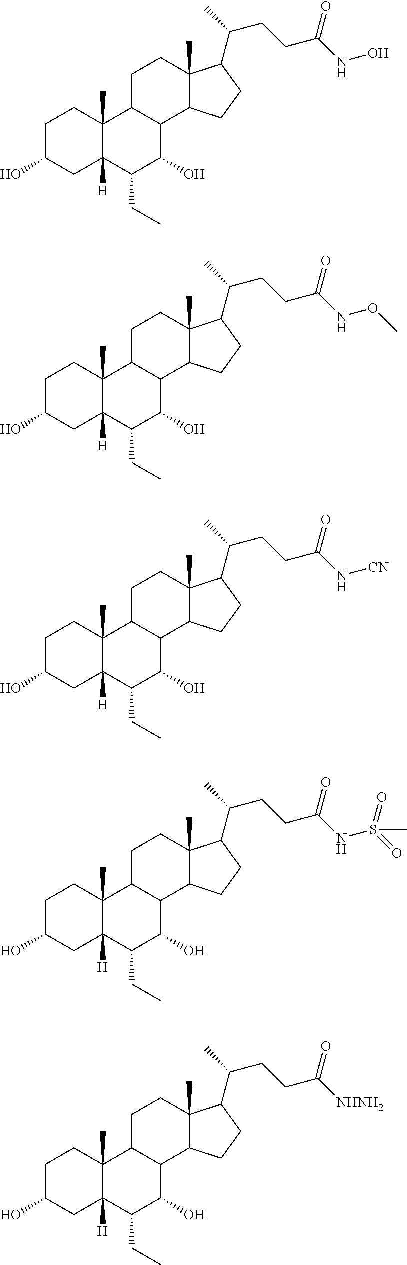 [SMILES: CC[C@H]1[C@@H](O)C2C3CCC([C@H](C)CCC(=O)NO)[C@@]3(C)CCC2[C@@]2(C)CC[C@@H](O)C[C@@H]12 ; CC[C@H]1[C@@H](O)C2C3CCC([C@H](C)CCC(=O)NOC)[C@@]3(C)CCC2[C@@]2(C)CC[C@@H](O)C[C@@H]12 ; CC[C@H]1[C@@H](O)C2C3CCC([C@H](C)CCC(=O)NC#N)[C@@]3(C)CCC2[C@@]2(C)CC[C@@H](O)C[C@@H]12 ; CC[C@H]1[C@@H](O)C2C3CCC([C@H](C)CCC(=O)NS(C)(=O)=O)[C@@]3(C)CCC2[C@@]2(C)CC[C@@H](O)C[C@@H]12 ; CC[C@H]1[C@@H](O)C2C3CCC([C@H](C)CCC(=O)NN)[C@@]3(C)CCC2[C@@]2(C)CC[C@@H](O)C[C@@H]12]

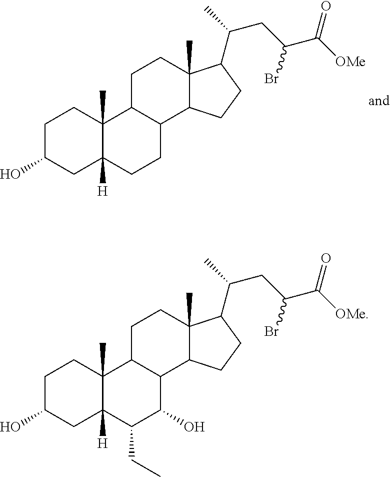 [SMILES: COC(=O)C(Br)C[C@@H](C)C1CCC2C3CC[C@@H]4C[C@H](O)CC[C@]4(C)C3CC[C@@]21C ; CC[C@H]1[C@@H](O)C2C3CCC([C@H](C)CC(Br)C(=O)OC)[C@@]3(C)CCC2[C@@]2(C)CC[C@@H](O)C[C@@H]12]

11. The cholic acid derivative, or the stereoisomer or pharmaceutically acceptable salt thereof according to claim 8, wherein the compound is selected from the group consisting of:

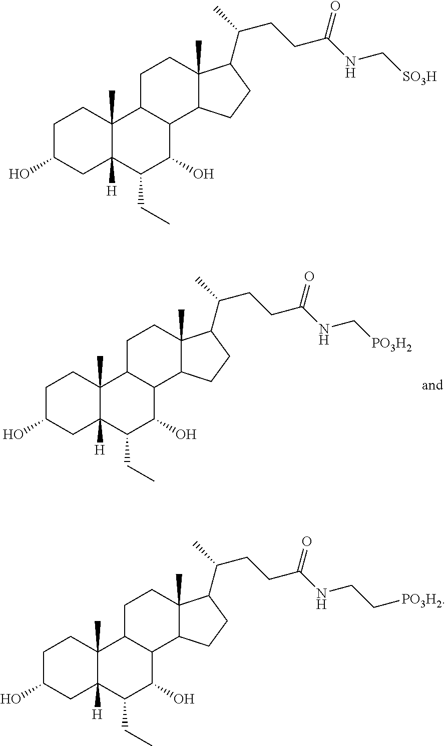

12. The cholic acid derivative, or the stereoisomer or pharmaceutically acceptable salt thereof according to claim 8, wherein the compound is:

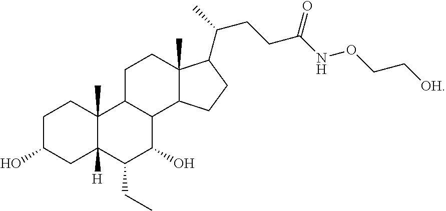 [SMILES: CC[C@H]1[C@@H](O)C2C3CCC([C@H](C)CCC(=O)NOCCO)[C@@]3(C)CCC2[C@@]2(C)CC[C@@H](O)C[C@@H]12]

13. The cholic acid derivative, or the stereoisomer or pharmaceutically acceptable salt thereof according to claim 8, wherein the compound is a compound of formula (V): 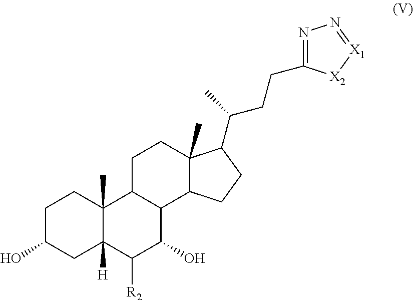
wherein X₁, X₂, R₂, R₈, R₉, R₁₀, R₁₁, R₁₂, R₁₃ and R₁₄ are as defined in claim 8.

14. The cholic acid derivative, or the stereoisomer or pharmaceutically acceptable salt thereof according to claim 8, wherein the compound is selected from the group consisting of:

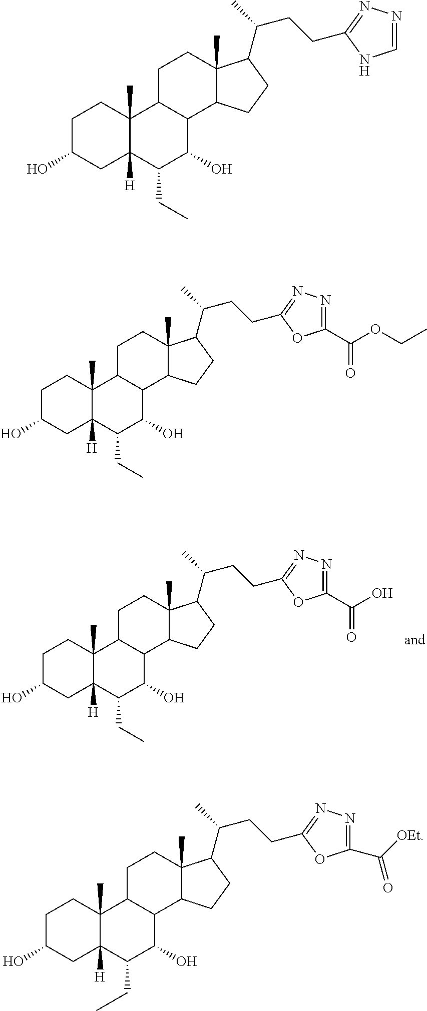 [SMILES: CC[C@H]1[C@@H](O)C2C3CCC([C@H](C)CCc4nnc[nH]4)[C@@]3(C)CCC2[C@@]2(C)CC[C@@H](O)C[C@@H]12 ; CCOC(=O)c1nnc(CC[C@@H](C)C2CCC3C4C(CC[C@@]32C)[C@@]2(C)CC[C@@H](O)C[C@H]2[C@@H](CC)[C@H]4O)o1 ; CC[C@H]1[C@@H](O)C2C3CCC([C@H](C)CCc4nnc(C(=O)O)o4)[C@@]3(C)CCC2[C@@]2(C)CC[C@@H](O)C[C@@H]12 ; CCOC(=O)c1nnc(CC[C@@H](C)C2CCC3C4C(CC[C@@]32C)[C@@]2(C)CC[C@@H](O)C[C@H]2[C@@H](CC)[C@H]4O)o1]

15. The cholic acid derivative, or the stereoisomer or pharmaceutically acceptable salt thereof according to claim 8, wherein the compound is a compound of formula (VI): 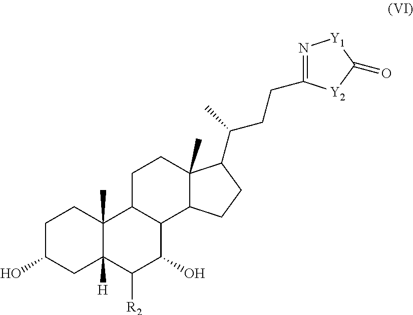
wherein Y₁, Y₂, R₂, R₈, R₉, R₁₀, R₁₁, R₁₂, R₁₃ and R₁₄ are as defined in claim 8.

16. The cholic acid derivative, or the stereoisomer or pharmaceutically acceptable salt thereof according to claim 8, wherein the compound is selected from the group consisting of:

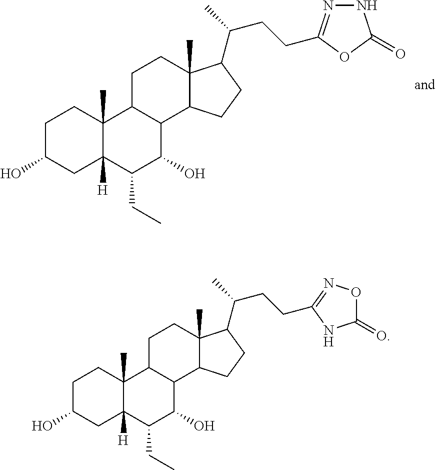 [SMILES: CC[C@H]1[C@@H](O)C2C3CCC([C@H](C)CCc4n[nH]c(=O)o4)[C@@]3(C)CCC2[C@@]2(C)CC[C@@H](O)C[C@@H]12 ; CC[C@H]1[C@@H](O)C2C3CCC([C@H](C)CCc4noc(=O)[nH]4)[C@@]3(C)CCC2[C@@]2(C)CC[C@@H](O)C[C@@H]12]

17. The cholic acid derivative, or the stereoisomer or pharmaceutically acceptable salt thereof according to claim 8, wherein the compound is a compound of formula (VII): 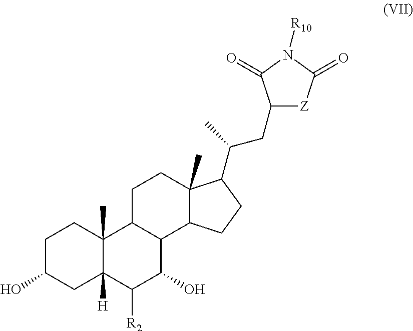
wherein R₂, R₈, R₉, R₁₀, R₁₁, R₁₂, R₁₃, R₁₄ and Z are as defined in claim 8.

18. The cholic acid derivative, or the stereoisomer or pharmaceutically acceptable salt thereof according to claim 8, wherein the compound is selected from the group consisting of:

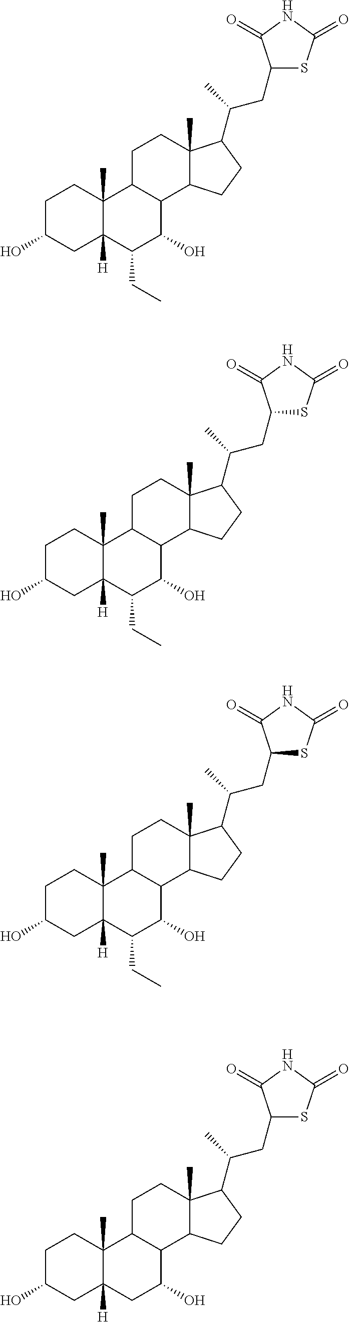 [SMILES: CC[C@H]1[C@@H](O)C2C3CCC([C@H](C)CC4SC(=O)NC4=O)[C@@]3(C)CCC2[C@@]2(C)CC[C@@H](O)C[C@@H]12 ; CC[C@H]1[C@@H](O)C2C3CCC([C@H](C)C[C@H]4SC(=O)NC4=O)[C@@]3(C)CCC2[C@@]2(C)CC[C@@H](O)C[C@@H]12 ; CC[C@H]1[C@@H](O)C2C3CCC([C@H](C)C[C@@H]4SC(=O)NC4=O)[C@@]3(C)CCC2[C@@]2(C)CC[C@@H](O)C[C@@H]12 ; C[C@H](CC1SC(=O)NC1=O)C1CCC2C3C(CC[C@@]21C)[C@@]1(C)CC[C@@H](O)C[C@H]1C[C@H]3O]

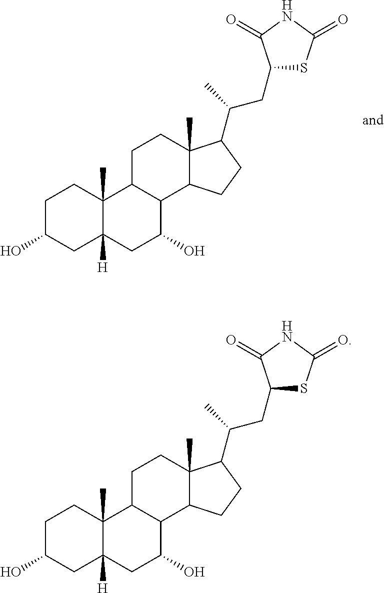 [SMILES: C[C@H](C[C@H]1SC(=O)NC1=O)C1CCC2C3C(CC[C@@]21C)[C@@]1(C)CC[C@@H](O)C[C@H]1C[C@H]3O ; C[C@H](C[C@@H]1SC(=O)NC1=O)C1CCC2C3C(CC[C@@]21C)[C@@]1(C)CC[C@@H](O)C[C@H]1C[C@H]3O]

19. A cholic acid derivative of formula (VIII), or a stereoisomer or a pharmaceutically acceptable salt thereof: 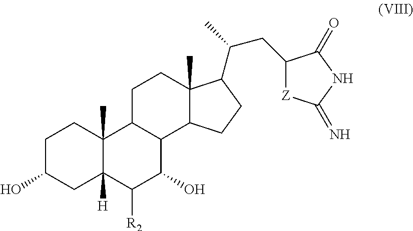
wherein Z is selected from the group consisting of oxygen and sulfur atom; and
R₂ is selected from the group consisting of hydrogen, fluorine, bromine, trifluoromethyl and C₁₋₈ alkyl.

20. The cholic acid derivative, or the stereoisomer or pharmaceutically acceptable salt thereof according to claim 19, wherein the compound is selected from the group consisting of:

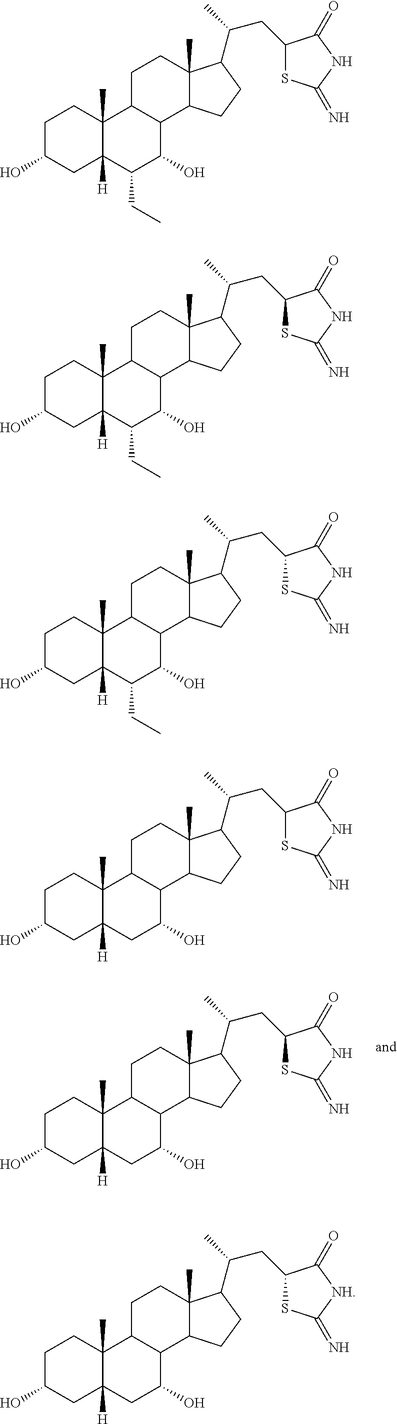

21. A process for preparing the cholic acid derivative, or the stereoisomer or pharmaceutically acceptable salt thereof according to claim 1, comprising the steps of: 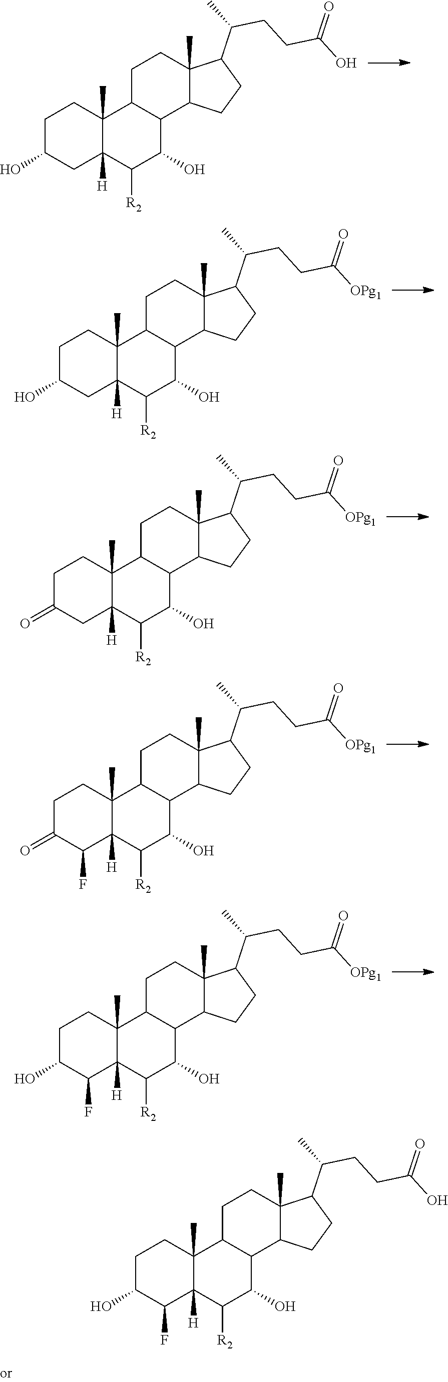 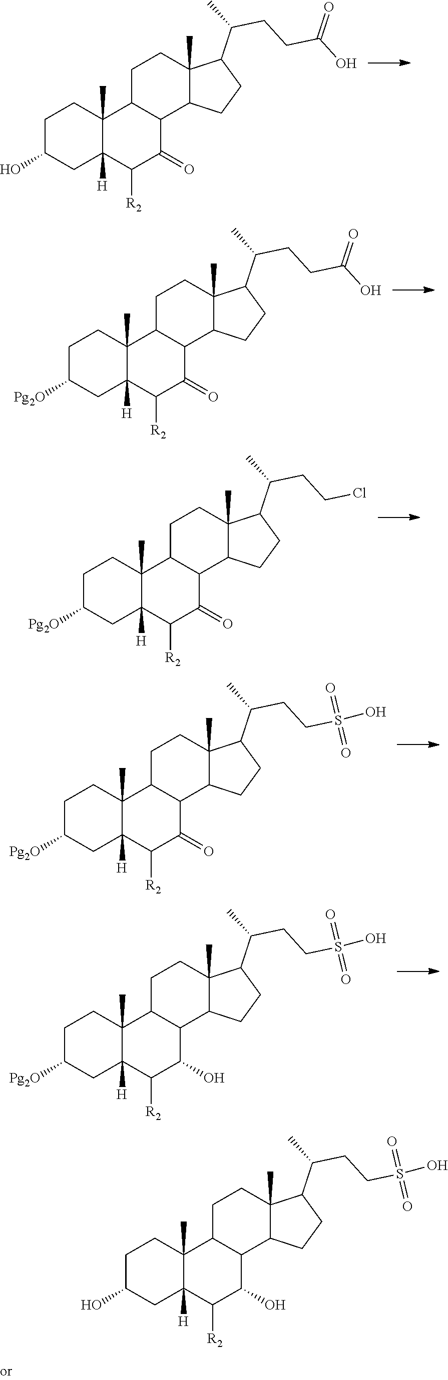 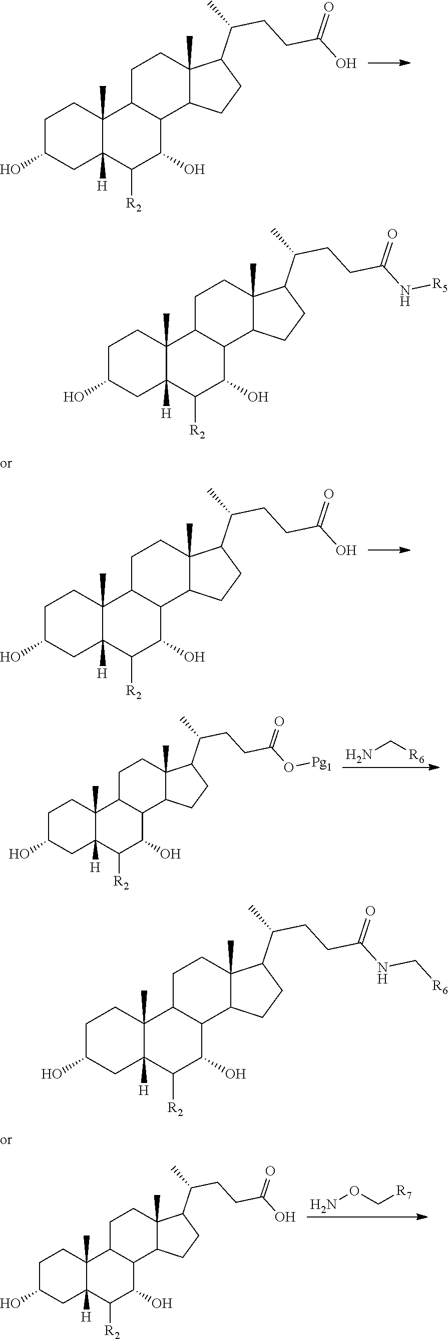 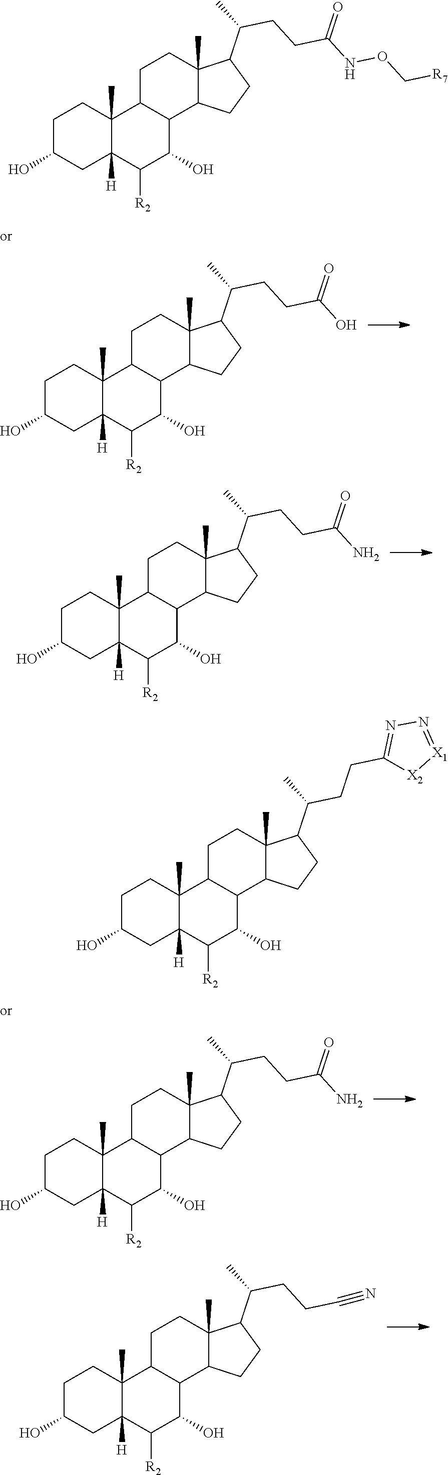 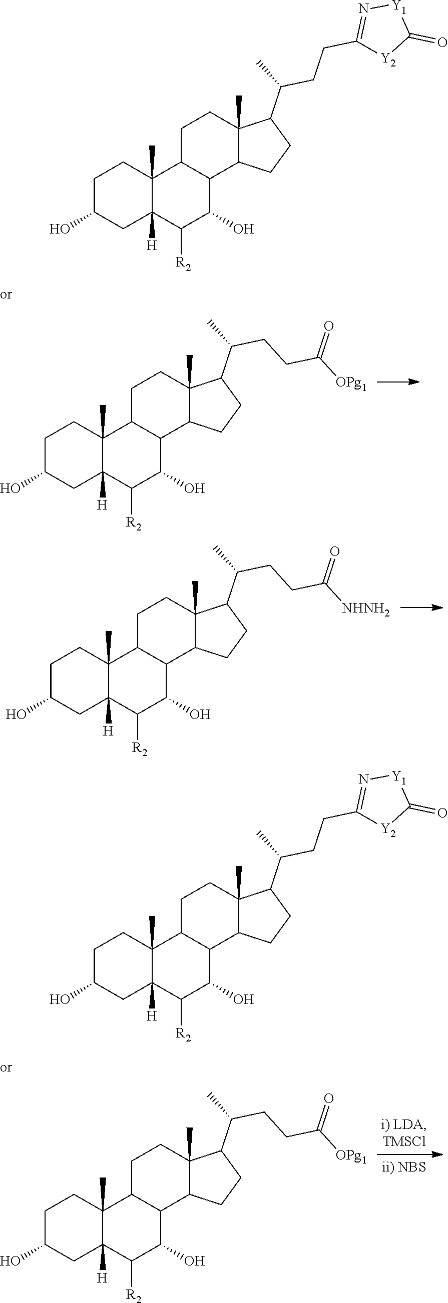 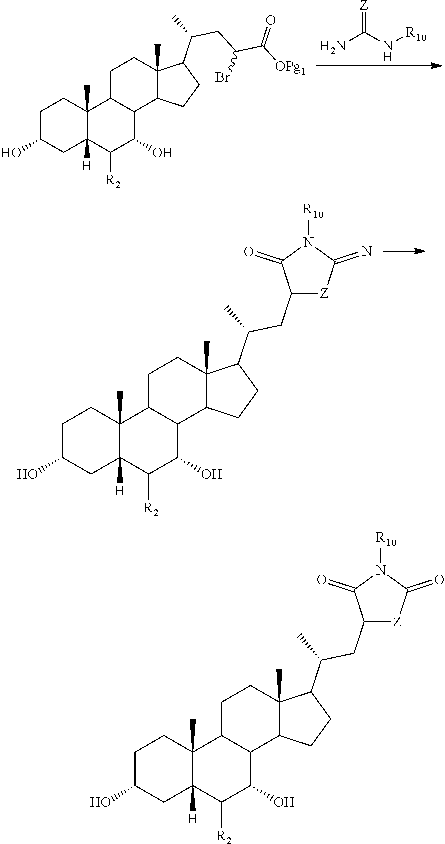
wherein R₂, R₅, R₆, R₇, R₈, R₉, R₁₀, R₁₁, R₁₂, R₁₃, R₁₄, X₁, X₂, Y₁, Y₂ and Z are as defined in claim 1; Pg₁ is a hydroxy protecting group; and Pg₂ is a hydroxy protecting group.

22. A pharmaceutical composition comprising a therapeutically effective amount of the cholic acid derivative, or the stereoisomer or pharmaceutically acceptable salt thereof according to claim 1, and a pharmaceutically acceptable carrier.

23.-25. (canceled)

26. A method to prevent or treat an FXR mediated disease or condition, comprising administering the pharmaceutical composition according to claim 22.
